(19) United States
(12) Patent Application Publication
(10) Pub. No.: US 2005/0043371 A1
(43) Pub. Date: Feb. 24, 2005

(54) Aminoalcohol derivatives and their use as beta 3 adrenergic agonists

(75) Inventors: Kiyoshi Taniguchi, Kobe-shi (JP); Naoaki Fujii, Osaka (JP); Minoru Sakurai, Osaka (JP); Yasuyo Tomishima, Osaka (JP); Hisashi Takasugi, Osaka (JP)

Correspondence Address: OBLON, SPIVAK, MCCLELLAND, MAIER & NEUSTADT, P.C., 1940 DUKE STREET, ALEXANDRIA, VA, 22314, US

(73) Assignee: Fujisawa Pharmaceutical Co. Ltd., Osaka (JP)

(21) Appl. No.: 10/959,171

(22) Filed: Oct. 7, 2004

Related U.S. Application Data
(63) Continuation of application No. 10/377,791, filed on Mar. 4, 2003, now abandoned.
Application No. 10/377,791 is a continuation of application No. 09/784,439, filed on Feb. 23, 2001, now abandoned.

(30) Foreign Application Priority Data
Aug. 26, 1999 (AU) PP5499

(51) Int. Cl. A61K 31/445; A61K 31/40; C07D 213/55; A61K 31/165; A61K 31/18; A61K 31/235

(52) U.S. Cl. 514/357; 514/408; 514/532; 514/602; 514/620; 546/335; 548/561; 560/38; 564/86

(57) ABSTRACT

This invention relates to new aminoalcohol derivatives or salts thereof represented by the following formula [I]: 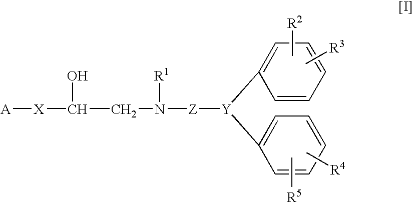 wherein each symbol is as defined in the specification or salts thereof which have gut selective sympathomimetic, anti-ulcerous, anti-pancreatitis, lipolytic, anti-urinary incontinence and anti-pollakiuria activities, to processes for the preparation thereof, to a pharmaceutical composition comprising the same and to a method for the prevention and/or treatment diseases indicated in the specification to a human being or an animal.

### TECHNICAL FIELD

This invention relates to new aminoalcohol derivatives and salts thereof which are β₃ adrenergic receptor agonists and useful as a medicament.

### DISCLOSURE OF INVENTION

This invention relates to new aminoalcohol derivatives which are β₃ adrenergic receptor agonists and salts thereof.

More particularly, it relates to new aminoalcohol derivatives and salts thereof which have gut selective sympathomimetic, anti-ulcerous, anti-pancreatitis, lipolytic, anti-urinary incontinence and anti-pollakiuria activities, to processes for the preparation thereof, to a pharmaceutical composition comprising the same and to a method of using the same therapeutically in the treatment and/or prevention of gastro-intestinal disorders caused by smooth muscle contractions in human beings or animal.

One object of this invention is to provide new and useful aminoalcohol derivatives and salts thereof which have gut selective sympathomimetic, anti-ulcerous, lipolytic, anti-urinary incontinence and anti-pollakiuria activities.

Another object of this invention is to provide processes for the preparation of said aminoalcohol derivatives and salts thereof.

A further object of this invention is to provide a pharmaceutical composition comprising, as an active ingredient, said aminoalcohol derivatives and salts thereof.

Still further object of this invention is to provide a therapeutical method for the treatment and/or prevention of aforesaid diseases in human beings or animals, using said aminoalcohol derivatives and salts thereof.

The object aminoalcohol derivatives of this invention are new and can be represented by the following general formula [I]: 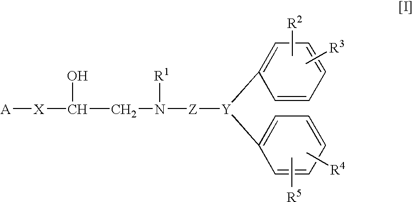
wherein
A is a heterocyclic group or aryl, each of which may have 1 to 3 same or different substituent(s) selected from a group consisting of halogen, hydroxy, amino, lower alkyl, lower alkylsulfonylamino, phenyl(lower)alkoxy and phenyl(lower)alkoxycarbonylamino,
—X— is bond, —CH₂—, —CH₂—CH₂—, —NH—CH₂—, —O—CH₂—, —S—CH₂—, —SO—CH₂— or —SO₂—CH₂—, 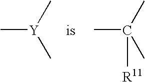
(in which R¹¹ is hydrogen, hydroxy, lower alkoxy or acyloxy) and 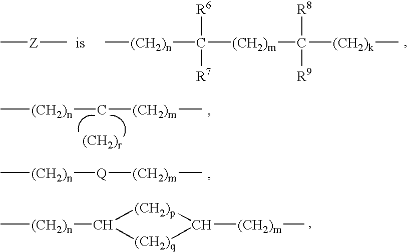
—(CH₂)ₙ—CH═CH—(CH₂)ₘ— or —(CH₂)ₙ—C═C—(CH₂)ₘ— (in which
   -Q- is —O—, —S—, —SO—, —SO₂—, 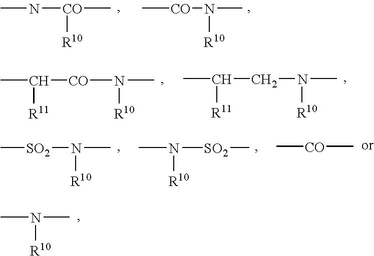
   wherein R¹⁰ is hydrogen or lower alkyl, and R¹¹ is lower alkyl,
   R⁶, R⁷, R⁸ and R⁹ are each independently hydrogen, hydroxy, lower alkyl, lower alkenyl, lower alkoxy, lower alkoxy(lower)alkyl or aryl which may have 1 to 3 lower alkoxy,
   n, m and k is each independently 0 to 6,
   p is 0 to 4,
   q is 1 to 4, and
   r is 2 to 7) and 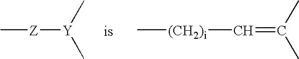
   (in which i is 0 to 6),
R¹ is hydrogen or an amino protective group, and
R², R³, R⁴ and R⁵ are each independently hydrogen;
   lower alkyl; lower alkylthio; lower alkylsulfonyl;
   hydroxy; lower alkoxy;
   amino; lower alkylamino; acylamino; N-(lower alkyl)acylamino; carboxy; lower alkoxycarbonyl;
   carbamoyl optionally substituted with one or two lower alkyl; hydroxy(lower)alkyl; lower alkoxy(lower)alkyl;
   N-acylamino(lower)alkyl; N-(lower alkyl)-N-acylamino(lower)alkyl; carboxy(lower)alkyl;
   lower alkoxycarbonyl(lower)alkyl; carbamoyl(lower)alkyl optionally substituted with one or two lower alkyl; or 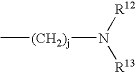
   (in which R¹² and R¹³ are each independently hydrogen or lower alkyl, or R¹² and R¹³ may be bonded to form a lower alkylene chain, and j is 0 to 6).

The object compound [I] or a salt thereof can be prepared by the following processes. 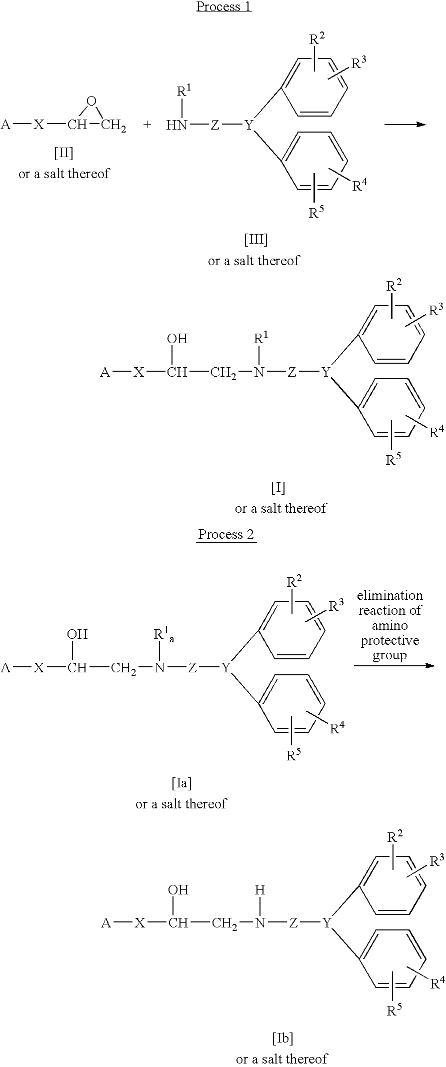
wherein A, X, Y, Z, R¹, R², R³, R⁴ and R⁵ are each as defined above, and
Rₐ¹ is an amino protective group.

In the above and subsequent description of the present specification, suitable examples of the various definition to be included within the scope of the invention are explained in detail in the following.

The term “lower” is intended to mean a group having 1 to 6 carbon atom(s), unless otherwise provided.

Suitable example of “lower alkyl” and “lower alkyl” moiety may include straight or branched one having 1 to 6 carbon atom(s), such as methyl, ethyl, propyl, isopropyl, butyl, isobutyl, sec-butyl, tert-butyl, pentyl, 1-methylpentyl, tert-pentyl, neo-pentyl, hexyl, isohexyl and the like.

Suitable “lower alkenyl” may include vinyl, 1-(or 2-)propenyl, 1-(or 2- or 3-)butenyl, 1-(or 2- or 3- or 4-)pentenyl, 1-(or 2- or 3- or 4- or 5-)hexenyl, methylvinyl, ethylvinyl, 1-(or 2- or 3-)methyl-1-(or 2-)propenyl, 1-(or 2- or 3-)ethyl-1-(or 2-)propenyl, 1-(or 2- or 3- or 4-)methyl-1-(or 2- or 3-)butenyl, and the like, in which more preferable example may be C₂-C₄ alkenyl.

Suitable “lower alkoxy” and “lower alkoxy” moiety may be a straight or branched one such as methoxy, ethoxy, propoxy, isopropoxy, 1-ethylpropoxy, butoxy, sec-butoxy, tert-butoxy, pentyloxy, neopentyloxy, tert-pentyloxy, hexyloxy, and the like, in which the preferred one may be C₁-C₄ alkoxy, and the most preferred one may be methoxy.

Suitable example of “halogen” may be fluoro, chloro, bromo and iodo.

Suitable example of “aryl” and “ar” moiety may include phenyl, naphthyl, anthryl, and the like, in which the preferred one may be phenyl.

Suitable example of “heterocyclic group” may include
unsaturated 3 to 8-membered (more preferably 5 or 6-membered) heteromonocyclic group containing 1 to 4 nitrogen atom(s), for example, pyrrolyl, pyrrolinyl, imidazolyl, pyrazolyl, pyridyl, dihydropyridyl, pyrimidyl, pyrazinyl, pyridazinyl, triazolyl (e.g., 4H-1,2,4-triazolyl, 1H-1,2,3-triazolyl, 2H-1,2,3-triazolyl, etc.), tetrazolyl (e.g., 1H-tetrazolyl, 2H-tetrazolyl, etc.), etc.;
saturated 3 to 8-membered (more preferably 5 or 6-membered) heteromonocyclic group containing 1 to 4 nitrogen atom(s), for example, pyrrolidinyl, imidazolidinyl, piperidyl, piperazinyl, etc.;
unsaturated condensed heterocyclic group containing 1 to 4 nitrogen atom(s), for example, indolyl, isoindolyl, indolinyl, indolizinyl, benzimidazolyl, quinolyl, isoquinolyl, indazolyl, benzotriazolyl, etc.;
unsaturated 3 to 8-membered (more preferably 5 or 6-membered) heteromonocyclic group containing 1 to 2 oxygen atom(s) and 1 to 3 nitrogen atom(s), for example, oxazolyl, isoxazolyl, oxadiazolyl (e.g., 1,2,4-oxadiazolyl, 1,3,4-oxadiazolyl, 1,2,5-oxadiazolyl, etc.), etc.;
saturated 3 to 8-membered (more preferably 5 or 6-membered) heteromonocyclic group containing 1 to 2 oxygen atom(s) and 1 to 3 nitrogen atom(s), for example, morpholinyl, sydnonyl, etc.;
unsaturated condensed heterocyclic group containing 1 to 2 oxygen atom(s) and 1 to 3 nitrogen atom(s), for example, benzoxazolyl, benzoxadiazolyl, etc.;
unsaturated 3 to 8-membered (more preferably 5 or 6-membered) heteromonocyclic group containing 1 to 2 sulfur atom(s) and 1 to 3 nitrogen atom(s), for example, thiazolyl, isothiazolyl, thiadiazolyl (e.g., 1,2,3-thiadiazolyl, 1,2,4-thiadiazolyl, 1,3,4-thiadiazolyl, 1,2,5-thiadiazolyl, etc.), dihydrothiazinyl, etc.;
saturated 3 to 8-membered (more preferably 5 or 6-membered) heteromonocyclic group containing 1 to 2 sulfur atom(s) and 1 to 3 nitrogen atom(s), for example, thiazolidinyl, etc.;
unsaturated 3 to 8-membered (more preferably 5 or 6-membered) heteromonocyclic group containing 1 to 2 sulfur atom(s), for example, thienyl, dihydrodithiinyl, dihydrodithionyl, etc.;
unsaturated condensed heterocyclic group containing 1 to 2 sulfur atom(s) and 1 to 3 nitrogen atom(s), for example, benzothiazolyl, benzothiadiazolyl, imidazothiadiazolyl, etc.;
unsaturated 3 to 8-membered (more preferably 5 or 6-membered) heteromonocyclic group containing an oxygen atom, for example, furyl, etc.;
saturated 3 to 8-membered (more preferably 5 or 6-membered) heteromonocyclic group containing an oxygen atom, for example, tetrahydrofuran, tetrahydropyran, etc.;
unsaturated 3 to 8-membered (more preferably 5 or 6-membered) heteromonocyclic group containing an oxygen atom and 1 to 2 sulfur atom(s), for example, dihydrooxathiinyl, etc.;
unsaturated condensed heterocyclic group containing 1 to 2 sulfur atom(s), for example, benzothienyl, benzodithiinyl, etc.;
unsaturated condensed heterocyclic group containing an oxygen atom and 1 to 2 sulfur atom(s), for example, benzoxathiinyl, etc.; 2-oxo-2,3-dihydro-1H-benzimidazolyl; and the like.

Suitable example of “amino protective group” moiety may be common amino protective group such as acyl, for example, substituted or unsubstituted lower alkanoyl [e.g. formyl, acetyl, propionyl, trifluoroacetyl, etc.], phthaloyl, lower alkoxycarbonyl [e.g. tert-butoxycarbonyl, tert-amyloxy-carbonyl, etc.], substituted or unsubstituted aralkyloxy-carbonyl [e.g. benzyloxycarbonyl, p-nitrobenzyloxycarbonyl, etc.], substituted or unsubstituted arenesulfonyl [e.g. benzenesulfonyl, tosyl, etc.], nitrophenylsulfenyl, ar(lower)alkyl [e.g. trityl, benzyl, etc.], and the like, in which preferable one is phenyl(lower)alkyl such as benzyl.

Suitable “acyl” and “acyl” moiety may be carboxy; esterified carboxy; carbamoyl optionally substituted with one or two lower alkyl; lower alkylsulfonyl [e.g. methylsulfonyl, ethylsulfonyl, propylsulfonyl, butylsulfonyl, pentylsulfonyl, hexylsulfonyl, etc.]; substituted or unsubstituted lower alkanoyl [e.g. formyl, acetyl, trifluoroacetyl, propanoyl, butanoyl, 2-methylpropanoyl, pentanoyl, 2,2-dimethylpropanoyl, hexanoyl, etc.]; and the like.

The esterified carboxy may be substituted or unsubstituted lower alkoxycarbonyl [e.g. methoxycarbonyl, ethoxycarbonyl, propoxycarbonyl, butoxycarbonyl, hexyloxycarbonyl, 2-iodoethoxycarbonyl, trifluoromethoxycarbonyl, 2,2,2-trichloroethoxycarbonyl, etc.], substituted or unsubstituted aryloxycarbonyl [e.g. phenoxycarbonyl, 4-nitrophenoxycarbonyl, 2-naphthyloxycarbonyl, etc.], substituted or unsubstituted ar(lower)alkoxycarbonyl [e.g. benzyloxycarbonyl, phenethyloxycarbonyl, benzhydryloxycarbonyl, 4-nitrobenzyloxycarbonyl, etc.] and the like, in which the preferred one may be lower alkoxycarbonyl, and the most preferred one may be ethoxycarbonyl.

Suitable example of “lower alkylsulfonylamino” may include methylsulfonylamino, ethylsulfonylamino, propylsulfonylamino, butylsulfonylamino, pentylsulfonylamino, hexylsulfonylamino, and the like, in which the preferred one may be (C₁-C₄)alkylsulfonylamino, and the most preferred one may be methylsulfonylamino.

Suitable example of “heterocyclic group” in A can be referred to aforementioned “heterocyclic group”, in which the preferred one may be unsaturated 3 to 8-membered (more preferably 5 or 6-membered) heteromonocyclic group containing 1 to 4 nitrogen atom(s) or unsaturated condensed heterocyclic group containing 1 to 4 nitrogen atom(s), and the most preferred one may be pyridyl, indolyl or 2-oxo-2,3-dihydro-1H-benzimidazolyl.

The lower alkylene chain formed by R⁷ and R⁸ is a straight or branched chain alkylene having 1 to 6 carbon atoms and is exemplified by methylene, ethylene, trimethylene, propylene, butylene, 1,2-dimethylethylene, pentamethylene and hexamethylene.

Preferred embodiments of the object compound [I] are as follow:
A is pyridyl, indolyl, 2-oxo-2,3-dihydro-1H-benzimidazolyl or phenyl, each of which may have 1 to 3 same or different substituent(s) selected from a group of hydroxy, amino lower alkyl (more preferably methyl), lower alkylsulfonylamino (more preferably methanesulfonylamino), phenyl(lower)alkoxy (more preferably benzyloxy) and phenyl(lower)alkoxycarbonylamino (more preferably benzyloxycarbonylamino),
—X— is bond, —CH₂—, —CH₂—CH₂—, —O—CH₂— or —SO₂—CH₂—, 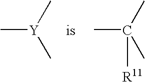
(in which R¹¹ is hydrogen or hydroxy) and 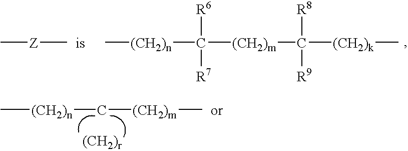
   (CH₂)ₙ-Q-(CH₂)ₘ— (in which
   -Q- is 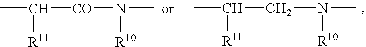
   wherein
      R¹⁰ is hydrogen or lower alkyl (more preferably methyl) and
      R¹¹ is lower alkyl (more preferably methyl),
   R⁶, R⁷, R⁸ and R⁹ are each independently hydrogen, lower alkyl (more preferably methyl) or aryl (more preferably phenyl) which may have 1 to 3 lower alkoxy (more preferably methoxy),
   n, m and k is each independently 0 to 6, and
   r is 2 to 7) and 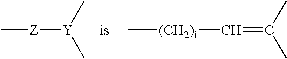
   (in which i is 0 to 6),
R¹ is hydrogen or ar(lower)alkyl (more preferably benzyl), and
R², R³, R⁴ and R⁵ are each independently hydrogen;
   lower alkyl (more preferably methyl); lower alkylthio (more preferably methylthio); lower alkylsulfonyl (more preferably methanesulfonyl); hydroxy; lower alkoxy (more preferably methoxy or ethoxy);
   amino; lower alkylamino (more preferably methylamino); acylamino (more preferably lower alkoxycarbonylamino, lower alkylsulfonylamino, lower alkanoylamino, ureido or trifluoroacetylamino, most preferably methoxycarbonylamino, ethoxycarbonylamino, methanesulfonylamino, formylamino, acetylamino or propionylamino); N-(lower alkyl)acylamino [more preferably N-(lower alkyl)-[(lower)alkoxycarbonyl]amino, most preferably N-methyl-methoxycarbonylamino]; carboxy; or lower alkoxycarbonyl (more preferably methoxycarbonyl).

More preferred embodiments of the object compound [I] are as follow:
A is phenyl each of which may have 1 to 3 same or different substituent(s) selected from a group of hydroxy, amino, lower alkylsulfonylamino (more preferably methanesulfonylamino) and phenyl(lower)alkoxy (more preferably benzyloxy),
—X— is bond, —CH₂—, —CH₂—CH₂—, —O—CH₂— or —SO₂—CH₂—, 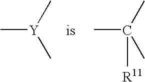
(in which R¹¹ is hydrogen or hydroxy),
-Z- is 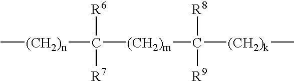
(in which
   R⁶, R⁷, R⁸ and R⁹ are each independently hydrogen, lower alkyl (more preferably methyl) or phenyl which may have 1 to 3 lower alkoxy (more preferably methoxy),
   n, m and k is each independently 0 or 1),
R¹ is hydrogen or phenyl(lower)alkyl (more preferebly benzyl), and
R², R³, R⁴ and R⁵ are each independently hydrogen; lower alkyl (more preferably methyl); lower alkylthio (more preferably methylthio); lower alkylsulfonyl (more preferably methanesulfonyl); hydroxy; lower alkoxy (more preferably methoxy or ethoxy); amino; lower alkylamino (more preferably methylamino); lower alkoxycarbonylamino (more preferably methoxycarbonylamino or ethoxycarbonylamino); lower alkylsulfonylamino (more preferably methanesulfonylamino); lower alkanoylamino (more preferably formylamino, acetylamino or propionylamino); ureido; trifluoroacetylamino; N-(lower alkyl)-[(lower)alkoxycarbonyl]amino (more preferably N-methyl-methoxycarbonylamino); carboxy; or lower alkoxycarbonyl (more preferably methoxycarbonyl).

Suitable salts of the object aminoalcohol derivatives [I] are pharmaceutically acceptable salts and include conventional non-toxic salts such as an inorganic acid addition salt [e.g. hydrochloride, hydrobromide, sulfate, phosphate, etc.], an organic acid addition salt [e.g. formate, acetate, trifluoroacetate, oxalate, maleate, fumarate, tartrate, methanesulfonate, benzenesulfonate, toluenesulfonate, etc.], an alkali metal salt [e.g. sodium salt, potassium salt, etc.] or the like.

The processes for preparing the object compound [I] are explained in detail in the following.

Process 1

The object compound [I] or a salt thereof can be prepared by reacting a compound [II] with a compound [III] or a salt thereof.

Suitable salt of the compound [III] may be the same as those exemplified for the compound [I].

The reaction is preferably carried out in the presence of a base such as an alkali metal carbonate [e.g. sodium carbonate, potassium carbonate, etc.], an alkaline earth metal carbonate [e.g. magnesium carbonate, calcium carbonate, etc.], an alkali metal bicarbonate [e.g. sodium bicarbonate, potassium bicarbonate, etc.], tri(lower)alkylamine [e.g. trimethylamine, triethylamine, etc.], picoline or the like.

The reaction is usually carried out in a conventional solvent, such as an alcohol [e.g. methanol, ethanol, propanol, isopropanol, etc.], diethyl ether, tetrahydrofuran, dioxane, or any other organic solvent which does not adversely influence the reaction.

The reaction temperature is not critical, and the reaction can be carried out under cooling to heating.

Process 2

The object compound [Ib] or a salt thereof can be prepared by subjecting a compound [Ia] or a salt thereof to elimination reaction of the amino protective group.

Suitable salts of the compounds [Ia] and [Ib] may be the same as those exemplified for the compound [I].

This reaction is carried out in accordance with a conventional method such as hydrolysis, reduction or the like.

The hydrolysis is preferably carried out in the presence of a base or an acid including Lewis acid.

Suitable base may include an inorganic base and an organic base such as an alkali metal [e.g. sodium, potassium, etc.], an alkaline earth metal [e.g. magnesium, calcium, etc.], the hydroxide or carbonate or bicarbonate thereof, hydrazine, trialkylamine [e.g. trimethylamine, triethylamine, etc.], picoline, 1,5-diazabicyclo[4.3.0]non-5-ene, 1,4-diazabicyclo[2.2.2]octane, 1,8-diazabicyclo[5.4.0]undec-7-ene, or the like.

Suitable acid may include an organic acid [e.g. formic acid, acetic acid, propionic acid, trichloroacetic acid, trifluoroacetic acid, etc.], an inorganic acid [e.g. hydrochloric acid, hydrobromic acid, sulfuric acid, hydrogen chloride, hydrogen bromide, hydrogen fluoride, etc.] and an acid addition salt compound [e.g. pyridine hydrochloride, etc.].

The elimination using trihaloacetic acid [e.g. trichloroacetic acid, trifluoroacetic acid, etc.] or the like is preferably carried out in the presence of cation trapping agents [e.g. anisole, phenol, etc.].

The reaction is usually carried out in a solvent such as water, an alcohol [e.g. methanol, ethanol, etc.], methylene chloride, chloroform, tetrachloromethane, tetrahydrofuran, a mixture thereof or any other solvent which does not adversely influence the reaction. A liquid base or acid can be also used as the solvent. The reaction temperature is not critical and the reaction is usually carried out under cooling to heating.

The reduction method applicable for the elimination reaction may include chemical reduction and catalytic reduction.

Suitable reducing agents to be used in chemical reduction are a combination of metal [e.g. tin, zinc, iron, etc.] or metallic compound [e.g. chromium chloride, chromium acetate, etc.] and an organic or inorganic acid [e.g. formic acid, acetic acid, propionic acid, trifluoroacetic acid, p-toluenesulfonic acid, hydrochloric acid, hydrobromic acid, etc.].

Suitable catalysts to be used in catalytic reduction are conventional ones such as platinum catalysts [e.g. platinum plate, spongy platinum, platinum black, colloidal platinum, platinum oxide, platinum wire, etc.], palladium catalysts [e.g. spongy palladium, palladium black, palladium oxide, palladium on carbon, colloidal palladium, palladium on barium sulfate, palladium on barium carbonate, etc.], nickel catalysts [e.g. reduced nickel, nickel oxide, Raney nickel, etc.], cobalt catalysts [e.g. reduced cobalt, Raney cobalt, etc.], iron catalysts [e.g. reduced iron, Raney iron, etc.], copper catalysts [e.g. reduced copper, Raney copper, Ullman copper, etc.] and the like.

In case that the amino protective group is benzyl, the reduction is preferably carried out in the presence of a combination of palladium catalysts [e.g. palladium black, palladium on carbon, etc.] and formic acid or its salt [e.g. ammonium formate, etc.].

The reduction is usually carried out in a conventional solvent which does not adversely influence the reaction such as water, an alcohol [e.g. methanol, ethanol, propanol, etc.], chlorobenzene, N,N-dimethylformamide, or a mixture thereof. Additionally, in case that the above-mentioned acids to be used in chemical reduction are in liquid, they can also be used as a solvent. Further, a suitable solvent to be used in catalytic reduction may be the above-mentioned solvent, and other conventional solvent such as diethyl ether, dioxane, tetrahydrofuran, etc. or a mixture thereof.

The reaction temperature of this reduction is not critical and the reaction is usually carried out under cooling to heating.

The compounds obtained by the above processes can be isolated and purified by a conventional method such as pulverization, recrystallization, column chromatography, reprecipitation, or the like, and converted to the desired salt in conventional manners, if necessary.

It is to be noted that the compound [I] and the other compounds may include one or more stereoisomers due to asymmetric carbon atoms, and all of such isomers and mixture thereof are included within the scope of this invention.

It is further to be noted that isomerization or rearrangement of the object compound [I] may occur due to the effect of the light acid, base or the like, and the compound obtained as the result of said isomerization or rearrangement is also included within the scope of the present invention.

It is also to be noted that the solvating form of the compound [I] (e.g. hydrate, etc.) and any form of the crystal of the compound [I] are included within the scope of the present invention.

The object compound [I] or a salt thereof possesses gut selective sympathomimetic, anti-ulcerous, anti-pancreatitis, lipolytic and anti-pollakiuria activities, and are useful for the treatment and/or prevention of gastro-intestinal disorders caused by smooth muscle contractions in human beings or animals, and more particularly to methods for the treatment and/or prevention of spasm or hyperanakinesia in case of irritable bowel syndrome, gastritis, gastric ulcer, duodenal ulcer, enteritis, cholecystopathy, cholangitis, urinary calculus and the like; for the treatment and/or prevention of ulcer such as gastric ulcer, duodenal ulcer, peptic ulcer, ulcer causes by non steroidal anti-inflammatory drugs, or the like; for the treatment and/or prevention of dysuria such as pollakiuria, urinary incontinence or the like in case of nervous pollakiuria, neurogenic bladder dysfunction, nocturia, unstable bladder, cystospasm, chronic cystitis, chronic prostatitis, prostatic hypertrophy or the like; and for the treatment and/or prevention of pancreatitis, obesity, diabetes, glycosuria, hyperlipidemia, hypertension, atherosclerosis, glaucoma, melancholia, depression or the like; and for the treatment and/or prevention of diseases as the result of insulin resistance (e.g. hypertension, hyperinsulinemia, etc.), and the like.

In order to show the usefulness of the compound [I] for the prophylactic anc therapeutic treatment of above-mentioned disease in human beings or animals, the pharmacological test data of a representative compound thereof are shown in the following.

Test

Effect on the increase in intravesical pressure induced by carbachol in anesthetized dog

Test Compouund

(1) (2S)-1-[[(2RS)-4,4-bis(4-hydroxyphenyl)-2-butyl]amino]-3-phenoxy-2-propanol hydrochloride

Test Method

Female Beagle dogs weighing 8.0-15.0 kg were fasted for 24 hours and maintained under halothane anesthesia. A 12F Foley catheter was lubricated with water soluble jelly, inserted into the urethral orifice and advanced approximately 10 cm until the balloon tip was placed well inside the bladder. The balloon was then inflated with 5 ml of room air and catheter slowly withdrawn just part the first resistance that is felt at the bladder neck. Urine was completely drained out through the catheter, and 30 ml of biological saline was infused. The catheter was connected to pressure transducer, and intravesical pressure was continuously recorded. The test compound was injected intravenously at 5 minutes before the administration of carbachol (1.8 μg/kg).

Test Results

| Treatment | Increase in intravescial pressure (mmHg) |
|---|---|
| Control | 4.5 ± 0.3 |
| Test Compound (1) | 2.5 ± 0.3** |
| (0.01 mg/kg) | |

**P < 0.01 vs Control (ANOVA) (N = 3)

The following Preparations and Examples are given for the purpose of illustrating this invention.

### EXAMPLE 1

A solution of (2S)-3-phenoxy-1,2-epoxypropane (195 mg) (IL FARMACO, 50 (10), 643 (1995)) and 4,4-bis(4-hydroxyphenyl)-2-butylamine (257 mg) in ethanol (2 ml) was stirred under reflux for 24 hours and evaporated in vacuo. The residue was chromatographed (chloroform-methanol) over silica gel (9 g) and the eluate was treated with 4N hydrogen chloride in ethyl acetate to afford a crude oil, which was powdered from diethyl ether to afford (2S)-1-[[(2RS)-4,4-bis(4-hydroxyphenyl)-2-butyl]amino]-3-phenoxy-2-propanol hydrochloride (117 mg) as a pale brown powder.

mp: 73° C. (dec.)

IR (Nujol): 3600-3100, 2700-2400, 1230 cm⁻¹

NMR (DMSO-d₆, δ): 1.21-1.27 (3H, m, CH₃), 1.94 (1H, m, CH₂), 2.60 (1H, m, CH₂), 2.85-3.2 (3H, m, CH₂NCH), 3.95 (1H, m, CHAr₂), 4.01-4.05 (2H, m, ArOCH₂), 4.16 (1H, m, CHOH), 5.85 (1H, br s, OH), 6.64-6.72 (4H, m, aromatic H), 6.92-7.16 (7H, m, aromatic H), 7.26-7.35 (2H, m, aromatic H), 8.62 (1H, br, NH), 8.92 (1H, br, HCl), 9.23 (1H, br s, OH), 9.28 (1H, br s, OH)

MS m/z 408 (M⁺+1)

### EXAMPLE 2

(2R)-N-Benzyl-4,4-bis(4-methoxyphenyl)-2-butylamine hydrochloride (412 mg) was converted to the corresponding free base in a usual manner. 1.0M Solution of tin(IV) chloride in dichloromethane (1.5 ml) was added dropwise to a stirred solution of the free base and (2S)-3-phenoxy-1,2-epoxypropane (225 mg) (IL FARMACO, 50 (10), 643 (1995)) in dichloromethane (4 ml) at −10-−5° C. under a nitrogen atmosphere over 10 minutes and the resulting mixture was stirred for 1.5 hours at the same temperature. The reaction mixture was poured into 1N hydrochloric acid and the mixture was stirred under ice cooling for 20 minutes. The organic layer was separated, washed with an aqueous solution of sodium fluoride and a saturated aqueous solution of sodium bicarbonate, dried over sodium sulfate, and evaporated in vacuo. The residue was chromatographed (ethyl acetate) over silica gel (11 g) and the eluate was treated with 4N hydrogen chloride in ethyl acetate to afford (2S)-1-[N-benzyl-[(2R)-4,4-bis(4-methoxyphenyl)-2-butyl]amino]-3-phenoxy-2-propanol hydrochloride (136 mg) as an oil.

IR (Film): 3292, 2850-2400, 1243 cm⁻¹

NMR (CDCl₃, δ) 1.27 and 1.47 (3H, each d, J=6.2 and 6.6 Hz), 2.08 (1H, m), 2.9-3.5 (4H, m), 3.68-4.0 (2H, m), 3.74, 3.75 and 3.76 (6H, each s), 4.02-4.08 (2H, m), 4.10-4.26 (1H, m), 4.3-4.6 (1H, m), 6.72-7.43 and 7.64-7.68 (18H, m)

MS m/z: 526 (M⁺+1)

### EXAMPLE 3

(2S)-N-Benzyl-4,4-bis(4-methoxyphenyl)-2-butylamine hydrochloride (412 mg) was converted to the corresponding free base in a usual manner. A solution of the free base and (2S)-3-phenoxy-1,2-epoxypropane (195 mg) (IL FARMACO, 50 (10), 643 (1995)) in ethanol (4 ml) was stirred under reflux for 10 hours, cooled to room temperature, and evaporated in vacuo. The residue was chromatographed (chloroform) over silica gel and the eluate was treated with 4N hydrogen chloride in ethyl acetate to afford (2S)-1-[N-benzyl-[(2S)-4,4-bis(4-methoxyphenyl)-2-butyl]amino]-3-phenoxy-2-propanol hydrochloride (549 mg) as an amorphous powder.

[α]²D⁴: −22.59° (c=0.54, MeOH)

IR (KBr) 3300 (br), 2850-2400, 1248 cm⁻¹

NMR (CDCl₃, δ): 1.41 and 1.56 (3H, each d, J=6.6 Hz), 1.64 and 2.05 (1H, m), 2.94-3.6 (4H, m), 3.74, 3.75, 3.76 and 3.77 (6H, each s), 3.87-3.96 (2H, m), 4.05-4.25 (3H, m), 4.5-4.65 and 4.8 (1H, m), 5.9 (1H, br), 6.69-6.98 (8H, m), 7.08-7.17 (4H, m), 7.22-7.41 (4H, m), 7.65-7.73 (2H, m)

MS m/z: 526 (M⁺+1)

### EXAMPLE 4

A mixture of (2S)-1-[N-benzyl-[(2R)-4,4-bis(4-methoxyphenyl)-2-butyl]amino]-3-phenoxy-2-propanol hydrochloride (96 mg) and 10% Pd/C (50% wet, 60 mg) in methanol (4 ml) was stirred at room temperature in the presence of hydrogen at an atmospheric pressure for 6 hours and filtered. The filtrate was evaporated in vacuo and the residue was partitioned between dichloromethane and an aqueous solution of sodium bicarbonate. The organic layer was separated, dried over sodium sulfate, and evaporated in vacuo. The residue was chromatographed (dichloromethane-methanol) over silica gel (2 g) to afford an oil, which was converted to the corresponding oxalate in a usual manner to afford (2S)-1-[[(2R)-4,4-bis(4-methoxyphenyl)-2-butyl]amino]-3-phenoxy-2-propanol oxalate (1:1) (26 mg) as a colorless powder.

mp: 121-123° C. (from diethyl ether)

IR (KBr): 3423 (br), 2850-2650, 1734, 1701, 1633, 1603, 1250 cm⁻¹

NMR (DMSO-d₆, δ): 1.23 (3H, d, J=6.3 Hz), 1.99 (1H, m), 2.58 (1H, m), 2.85-3.01 (2H, m), 3.11-3.17 (1H, m), 3.69 (3H, s), 3.71 (3H, s), 3.9-4.6 (8H, m), 6.81-6.99 (7H, m), 7.16-7.35 (6H, m)

MS m/z 436 (M++1)

### EXAMPLE 5

The following compound was obtained according to a similar manner to that of Example 4.

(2S)-1-[[(2S)-4,4-Bis(4-methoxyphenyl)-2-butyl]amino]-3-phenoxy-2-propanol oxalate (1:1)

[α]_{D}²⁴: 11.06° (c=0.515, MeOH)

mp 79-94° C. (from diethyl ether)

IR (KBr) 3423 (br), 2750-2400, 1739-1691 (m), 1643, 1608, 1247 cm⁻¹

NMR (DMSO-d₆, δ): 1.25 (3H, d, J=6.3 Hz), 1.96 (1H, m), 2.66 (1H, m), 2.86 (1H, m) 2.95-3.15 (2H, m), 3.69 (3H, s), 3.70 (3H, s), 3.94-4.15 (4H, m), 5.1 (4H, br), 6.81-6.88 (4H, m), 6.92-6.99 (3H, m), 7.17-7.35 (6H, m)

MS m/z: 436 (M⁺+1)

Preparation 1

A mixture of methyl 3-aminobutyrate (4.3 g), (2S)-3-phenoxy-1,2-epoxypropane (4.59 g), ytterbium(III) trifluoromethanesulfonate (1.8 g) and dichloromethane (25 ml) was stirred at 40° C. for 2 hours and at room temperature overnight, worked up in the usual manner and purified by silica gel column chromatography (toluene:ethanol: concentrated ammonia water=9:1:0.1) to give (3RS)-3-[((2S)-2-hydroxy-3-phenoxypropyl)amino]butyric acid methyl ester (2.59 g).

IR (Neat): 3400 (br m), 1734 (s), 1599 (m), 1495 (m), 1458 (m), 1298 (m), 1246 (s), 1041 (m), 756 (m) cm⁻¹

NMR (CDCl₃, δ): 1.16 (3H, d, J=5.2 Hz), 2.41-2.46 (2H, m), 2.6-3.0 (2H, m), 3.14 (1H, quartet, J=6.4 Hz), 3.68 (3H, s), 3.9-4.1 (3H, m), 6.90-6.99 (3H, m), 7.24-7.33 (2H, m)

MS m/z: 268 (M⁺+1)

Preparation 2

A mixture of (2S)-N-benzyl-(2-hydroxy-3-phenoxypropyl)amine (142 mg), 5-bromopentanoic acid ethyl ester (173 mg), potassium carbonate (153 mg) and N,N-dimethylformamide (2 ml) was stirred at 80° C. for 4.5 hours, worked up in the usual manner and purified by silica gel column chromatography (20% ethyl acetate-hexane) to give (2S)-5-[N-benzyl-(2-hydroxy-3-phenoxypropyl)amino]pentanoic acid ethyl ester (93 mg).

NMR (CDCl₃, δ): 1.25 (3H, t, J=7.1 Hz), 1.5-1.7 (5H, broad), 2.2-2.4 (2H, m), 2.4-2.8 (4H, m), 3.5-3.7 (1H, m), 3.7-3.9 (1H, m), 3.94 (2H, t, J=3.9 Hz), 4.0-4.2 (1H, m), 4.12 (2H, quartet, J=7.1 Hz), 6.86-6.98 (4H, m), 7.2-7.4 (6H, m)

MS m/z: 386 (M⁺+1)

Preparation 3

The following compound was obtained according to a similar manner to that of Preparation 2.

(2S)-4-[N-Benzyl-(2-hydroxy-3-phenoxypropyl)amino]-butanoic acid methyl ester

IR (Neat): 3482 (br m), 2949 (m), 1736 (s), 1599 (w), 1495 (m), 1456 (m), 1246 (s), 1041 (m), 754 (m), 696 (m) cm⁻¹

NMR (CDCl₃, δ): 1.88 (2H, quintet, J=7.1 Hz), 2.32 (2H, t, J=7.2 Hz), 2.5-2.8 (4H, m), 3.5-3.7 (1H, m), 3.64 (3H, s), 3.84 (1H, d, J=13.0 Hz), 3.89-4.0 (2H, m), 4.0-4.2 (1H, m), 6.86-6.99 (4H, m), 7.2-7.4 (6H, m)

MS m/z: 358 (M⁺+1)

Preparation 4

The following compounds were obtained according to a similar manner to that of Example 6.

(1) 1-[2,2-Bis(4-methoxyphenyl)-2-hydroxyethyl]cyclopentanol

IR (KBr): 3347 (s), 3240 (m), 2958 (s), 1608 (m), 1510 (s), 1466 (m), 1248 (s), 1174 (m), 1034 (s), 835 (m) cm⁻¹

NMR (CDCl₃, δ): 1.2-1.7 (8H, m), 2.39 (1H, br s), 2.70 (2H, s), 3.78 (6H, s), 4.87 (1H, br s), 6.82 (4H, d, J=8.9 Hz), 7.37 (4H, d, J=8.9 Hz)

(2) 3-(Dibenzylamino)-1,1-bis(4-bromophenyl)-1-propanol

NMR (CDCl₃, δ): 2.3-2.4 (2H, m), 2.6-2.7 (2H, m), 3.51 (4H, s), 7.07 (2H, d, J=8.5 Hz), 7.1-7.4 (16H, m)

MS m/z: 564, 566 (M⁺+1), 568

(3) (3RS)-3-(Dibenzylamino)-1,1-bis(4-bromophenyl)-1-butanol

NMR (CDCl₃, δ): 1.10 (3H, d, J=6.7 Hz), 2.09 (1H, d, J=14.8 Hz), 2.45 (1H, dd, J=11.2 and 14.8 Hz), 3.1-3.3 (1H, m), 3.18 (2H, d, J=12.8 Hz), 3.91 (2H, d, J=12.7 Hz), 6.82 (2H, d, J=8.7 Hz), 7.11 (2H, d, J=8.7 Hz), 7.2-7.3 (12H, m), 7.40 (2H, d, J=9.4 Hz)

(4) (2S)-1-Phenoxy-3-[N-benzyl-[(3RS)-1,1-bis(4-bromophenyl)-1-hydroxy-3-butyl]amino]-2-propanol

MS m/z: 638, 640 (M⁺+1), 642

(5) N-Benzyl-[4,4-bis(4-methoxyphenyl)butyl]amine

NMR (CDCl₃, δ): 1.4-1.7 (2H, m), 2.00 (2H, quartet, J=7.8 Hz), 2.64 (2H, t, J=7.1 Hz), 3.76 (6H, s), 3.72-3.79 (3H, m), 6.80 (4H, d, J=8.7 Hz), 7.11 (4H, d, J=8.7 Hz), 7.28 (5H, s)

MS m/z: 376 (M⁺+1)

(6) N-Benzyl-[3,3-bis(4-ethoxyphenyl)propyl]amine

MS m/z: 390 (M⁺+1)

Preparation 5

1-[2,2-Bis(4-methoxyphenyl)-2-hydroxyethyl]cyclopentanol (0.79 g) was hydrogenated in the usual manner to give 1-[2,2-bis(4-methoxyphenyl)ethyl]cyclopentanol (0.76 g).

IR (Neat): 3563 (m), 3448 (m), 2956 (s), 1610 (w), 1510 (s)., 1460 (m), 1246 (s), 1178 (m), 1036 (s), 829 (m) cm⁻¹

NMR (CDCl₃, δ): 1.5-1.9 (9H, m), 2.40 (2H, d, J=7.3 Hz), 3.77 (6H, s), 4.17 (1H, t, J=7.2 Hz), 6.81 (4H, d, J=6.6 Hz), 7.21 (4H, d, J=6.6 Hz)

Preparation 6

To a mixture of 1-[2,2-bis(4-methoxyphenyl)ethyl]-cyclopentanol (0.76 g), azidotrimethylsilane (0.32 g) and benzene (5 ml), boron trifluoride diethyl etherate (0.34 ml) was added dropwise at 0° C. The reaction mixture was stirred at room temperature for 30 minutes and worked up in the usual manner. The crude product was hydrogenated in the usual manner to give [1-[2,2-bis(4-methoxyphenyl)ethyl]-cyclopentyl]amine (0.74 g).

IR (Neat): 2949 (s), 1610 (m), 1510 (s), 1460 (m), 1248 (s), 1178 (m), 1038 (s), 829 (s) cm⁻¹

NMR (CDCl₃, δ): 1.3-1.8 (10H, m), 1.20 (2H, t, J=6.8 Hz), 3.77 (6H, s), 4.09 (1H, quartet, J=6.8 Hz), 6.81 (4H, dd, J=2.2 and 6.6 Hz), 7.14 (4H, d, J=6.5 Hz)

MS m/z: 326 (M⁺+1)

Preparation 7

To a solution of dibenzylamine (1.14 g) and tetrahydrofuran (4 ml), butyllithium (1.54M in hexane, 3.7 ml) was added dropwise at −78° C. under a flow of nitrogen. After 30 minutes, a solution of 3-(3,4-dimethoxyphenyl)-acrylic acid methyl ester (1.06 g) in tetrahydrofuran (3 ml) was added dropwise, stirred for 1 hour and worked up in the usual manner. The crude product was purified by silica gel column chromatography (hexane:ethyl acetate=5:1) to give 3-(dibenzylamino)-3-(3,4-dimethoxyphenyl)propionic acid methyl ester (0.53 g).

IR (Neat): 1739. (s), 1514 (s), 1458-(m), 1261 (m), 1146 (m), 1028. (m), 744 (m) cm⁻¹

NMR (CDCl₃, δ): 2.72 (1H, dd, J=7.3 and 14.4 Hz), 3.07 (1H, dd, J=8.6 and 14.4 Hz), 3.21 (1H, d, J=13.7 Hz), 3.65 (3H, s), 3.73 (2H, s), 3.79 (2H, s), 3.89 (6H, s), 4.25 (1H, t, J=7.4 Hz), 6.75-6.90 (3H, m), 7.1-7.4 (10H, m)

Preparation 8

A mixture of 3-(dibenzylamino)-3-(3,4-dimethoxyphenyl)-propionic acid methyl ester (0.53 g), acetic acid (3 ml), methanol (3 ml), and 20% palladium hydroxide on charcoal (0.05 g) was stirred under hydrogen (1 atm) at room temperature for 6 hours, filtered and evaporated to give 3-amino-3-(3,4-dimethoxyphenyl)propionic acid methyl ester acetate (0.24 g).

IR (KBr): 1729 (s), 1539 (s), 1523 (s), 1398 (m), 1265 (m), 1203 (m), 1155 (m), 1020 (m) cm⁻¹

NMR (MeOH-d₄, δ): 1.90 (3H, s), 2.92 (2H, dd, J=5.4 and 6.6 Hz), 3.63 (3H, s), 3.82 (3H, s), 3.84 (3H, s), 4.52 (1H, t, J=7.5 Hz), 6.95 (2H, s), 7.02 (1H, s)

Preparation 9

To a solution of 3-aminopropionic acid methyl ester hydrochloride (1.12 g) in methanol (10 ml), 28% sodium methoxide-methanol solution (1.60 g) was added, filtered and evaporated. To the crude product, (2S)-2-phenoxy-1,2-epoxypropane (901 mg) and methanol (10 ml) were added and stirred under reflux for 2.5 hours. The reaction mixture was evaporated and purified by silica gel column chromatography to give 3-[((2S)-2-hydroxy-3-phenoxypropyl)amino]propionic acid methyl ester (0.76 g).

IR (KBr): 1734 (s), 1601 (m), 1498 (m), 1435 (m), 1252 (s), 1196 (m), 1043 (m), 752 (m) cm⁻¹

NMR (CDCl₃, δ): 2.54 (2H, t, J=6.4 Hz), 2.72-2.98 (4H, m), 3.69 (3H, s), 3.97-4.07 (3H, m), 6.90-6.99 (3H, m), 7.25-7.32 (2H, m)

MS m/z: 254 (M⁺+1)

Preparation 10

The following compound was obtained according to a similar manner to that of Preparation 9.

(3RS)-3-[((2S)-2-Hydroxy-3-phenoxypropyl)amino]-3-phenylpropionic acid methyl ester

IR (KBr): 1724 (s), 1599 (m), 1495 (m), 1435 (m), 1246 (s), 1126 (m), 1038 (m), 756 (m), 698 (m) cm⁻¹

NMR (CDCl₃, δ): 2.54-2.75 (4H, m), 3.66 (1.5H, s), 3.67 (1.5H, s), 3.9-4.0 (2H, m), 4.0-4.2 (2H, m), 6.85-6.98 (3H, m), 7.2-7.4 (7H, m)

MS m/z: 330 (M⁺+1)

Preparation 11

To a mixture of N-carbobenzyloxy-D-alanine (0.81 g), [bis(4-methoxyphenyl)methyl]amine (0.80 g), 1-hydroxybenzotriazole (0.58 g) and N,N-dimethylformamide (5 ml), 1-ethyl-3-(3-dimethylaminopropyl)carbodiimide hydrochloride (0.76 g) was added at 0° C. and stirred at room temperature for 30 minutes. After a usual workup, N-carbobenzyloxy-D-alanine [bis(4-methoxyphenyl)methyl]amide (1.38 g) was obtained.

IR (KBr): 3296 (s), 1689 (m), 1647 (s), 1539 (s), 1512 (s), 0.1257 (s), 1176 (m), 1031 (m) cm⁻¹

NMR (DMSO-d₆, δ): 1.21 (3H, d, J=7.1 Hz), 3.33 (6H, s), 4.17 (1H, t, J=7.2 Hz), 5.01 (2H, s), 5.96 (1H, d, J=8.4 Hz), 6.86 (4H, d, J=8.7 Hz), 7.1-7.2 (4H, m), 7.3-7.5 (5H, m), 8.60 (1H, d, J=8.5 Hz)

Preparation 12

To a solution of N-carbobenzyloxy-D-alanine [bis(4-methoxyphenyl)methyl]amide (0.33 g) in methanol and tetrahydrofuran (1:2, 10 ml), 50% wet 10% palladium on charcoal was added and stirred under hydrogen atmosphere (1 atm) for 30 minutes. After a usual workup, D-alanine [bis(4-methoxyphenyl)methyl]amide (0.25 g) was obtained.

IR (Neat): 3357 (br s), 1678 (s), 1649 (s), 1538 (s), 1513 (s), 1259 (m), 1176 (m), 1032 (s), 831 (m), 812 (m) cm⁻¹

NMR (DMSO-d₆, δ): 1.13 (3H, d, J=6.9 Hz), 3.33 (6H, s), 3.3-3.4 (3H, br), 5.96 (1H, d, J=8.2 Hz), 6.87 (4H, d, J=8.7 Hz), 7.15 (4H, d, J=8.4 Hz), 8.44 (1H, d, J=8 Hz)

Preparation 13

To a solution of 4-methoxyphenylmagnesium bromide (1M in tetrahydrofuran, 35 ml) a solution of 3-(dibenzylamino)-propionic acid ethyl ester (4.87 g) in terahydrofuran (2 ml) was added, stirred under reflux for 1 hour, worked up in the usual manner and purified by silica gel column chromatography (hexane:ethyl acetate=5:1) to give 3-dibenzylamino-1,1-bis(4-methoxyphenyl)-1-propanol (3.45 g).

Preparation 14

3-(Dibenzylamino)-1,1-bis(4-methoxyphenyl)-1-propanol (2.0 g) was hydrogenated by the usual manner to give N-benzyl-[3,3-bis(4-methoxyphenyl)propyl]amine, which was further hydrogenated by heating with 20% palladium on charcoal and ammonium formate in methanol to give [3,3-bis(4-methoxyphenyl)propyl]amine (165 g).

Preparation 15

To a solution of 4-benzyloxy-3-nitrophenyl acetate (4.20 g) in methanol (20 ml), 28% sodium methoxide-methanol solution (2.96 g) was added and evaporated. To the crude residue, N,N-dimethylformamide (20 ml) and (2S)-3-[(3-nitrophenyl)sulfonyloxy]-1,2-epoxypropane (3.80 g) were added. The mixture was stirred at room temperature overnight, and worked up in the usual manner to give (2S)-3-(4-benzyloxy-3-nitrophenoxy)-1,2-epoxypropane (4.30 g).

NMR (CDCl₃, δ): 2.72-2.77 (1H, m), 2.92 (1H, quintets J=4.8 Hz), 3.32-3.37 (1H, m), 3.91 (1H, quartet, J=5.9 Hz), 4.27 (1H, dd, J=2.8 and 11.4 Hz), 5.18 (2H, s), 7.07-7.15 (2H, m), 7.34-7.46 (6H, m)

Preparation 16

A mixture of 3-(dibenzylamino)-1,1-bis(4-bromophenyl)-1-propanol (8.42 g), benzophenone imine (10.8 g), tris(dibenzylideneacetone)dipalladium (546 mg), (RS)-2,2′-bis(diphenylphosphino)-1,1′-binaphthyl (1.11 g), sodium tert-butoxide (5.7 g) and toluene (90 ml) was stirred at 100° C. for 6 hours. The reaction mixture was added to tetrahydrofuran (300 ml), and 3N hydrochloric acid (300 ml) and stirred at room temperature for 1.5 hours. The aqueous phase was separated, neutralized by sodium hydroxide and extracted with ethyl acetate. The ethyl acetate solution was evaporated and purified by silica gel column chromatography (hexane:ethyl acetate=1:1) to give 3-(dibenzylamino)-1,1-bis(4-aminophenyl)-1-propanol (1.76 g).

MS m/z: 438 (M⁺+1)

Preparation 17

The following compound was obtained according to a similar manner to that of Preparation 16.

(3RS)-3-(Dibenzylamino)-1,1-bis(4-aminophenyl)-1-butanol

IR (Neat): 3356 (m), 3219 (m), 2964 (m), 1622 (s), 1512 (s), 1454 (m), 1383 (w), 1271 (m), 1246 (m), 1176 (m), 1142 (m), 831 (m) cm⁻¹

NMR (CDCl₃, δ): 1.03 (3H, d, J=6.7 Hz), 2.02 (1H, d, J=11.7 Hz), 2.50 (1H, dd, J=11.2 and 14.7 Hz), 3.10-3.2 (1H, m), 3.21 (2H, d, J=13.0 Hz), 3.92 (2H, d, J=12.9 Hz), 6.35 (2H, d, J=6.5 Hz), 6.55 (2H, d, J=6.6 Hz), 6.76 (2H, d, J=6.6 Hz), 7.13 (2H, d, J=6.5 Hz), 7.24 (10H, s)

MS m/z: 452 (M⁺+1)

Preparation 18

To a mixture of 3-(dibenzylamino)-1,1-bis(4-aminophenyl)-1-propanol (0.64 g), pyridine (0.5 ml) and dichloromethane (10 ml), methyl chlorocarbonate (0.34 ml) was added at 0° C. and the reaction mixture was worked up in a usual manner. The crude product was dissolved in methanol (10 ml), followed by addition of 4N hydrogen chloride in 1,4-dioxane (0.5 ml) and 20% palladium hydroxide on charcoal. The mixture was stirred under hydrogen (1 atm) at room temperature overnight, worked up in a usual manner and purified by silica gel column chromatography (dichloromethane:methanol:concentrated ammonia water=20:1:0.1) to give N-benzyl-[3,3-bis[4-[(methoxycarbonyl)-amino]phenyl]propyl]amine (466 mg).

MS m/z: 448 (M⁺+1)

Preparation 19

A mixture of (3RS)-3-aminobutyric acid methyl ester hydrochloride (5.0 g), benzyl bromide (11.7 g), potassium carbonate (18 g) and N,N-dimethylformamide (40 ml) was stirred at room temperature overnight, worked up in the usual manner and purified by silica gel column chromatography (hexane:ethyl acetate=15:1) to give (3RS)-3-(dibenzylamino)butyric acid methyl ester (7.27 g).

IR (Neat): 2968 (m), 1741 (s), 1454 (m) 1259 (m), 1196 (m), 1146 (m), 1072 (m), 1022 (m), 744 (m), 698 (m) cm⁻¹

NMR (CDCl₃, δ): 1.10 (3H, d, J=6.7 Hz), 2.28 (1H, dd, J=6.9 and 13.9 Hz), 2.64 (1H, dd, J=8.01 and 13.9 Hz), 3.30 (1H, dd, J=6.8 and 14.7 Hz), 3.44 (2H, d, J=13.7 Hz), 3.60 (3H, s), 3.66 (2H, d, J=13.7 Hz), 7.18-7.35 (10H, m)

MS m/z: 298 (M⁺+1)

Preparation 20

The following compound was obtained according to a similar manner to that of Preparation 19.

(3RS)-3-[N-Benzyl-((2S)-2-hydroxy-3-phenoxypropyl)-amino]butyric acid methyl ester

MS m/z: 358 (M⁺+1)

Preparation 21

To a mixture of (3RS)-3-(dibenzylamino)-1,1-bis(4-aminophenyl)-1-butanol (360 mg), triethylamine (0.44 ml) and dichloromethane (4 ml), methanesulfonyl chloride (0.20 ml) was added dropwise at 0° C. The reaction mixture was stirred at room temperature for 30 minutes and worked up in the usual manner. The crude product was dissolved in methanol followed by addition of 10% palladium on charcoal (50% wet) and ammonium formate, heated under reflux for 1.5 hours, filtrated and extracted by water. The aqueous solution was treated with benzyl chlorocarbonate (136 μl) in the usual Shotten method, worked up in the usual manner and purified by silica gel column chromatography (hexane:ethyl acetate=2:3) to give (3RS)-[3,3-bis[4-(methanesulfonylamino)phenyl]-1-methylpropyl]carbamic acid benzyl ester (138 mg).

IR (Neat): 3259(m), 1680 (s), 1512 (s), 1331 (m), 1153 (s), 974 (m) cm⁻¹

NMR (CDCl₃, δ): 1.13 (3H, d, J=6.8 Hz), 2.4-2.6 (2H, m), 2.98 (6H, s), 3.7-3.9 (1H, m), 4.6-4.8 (1H, m), 6.52 (2H, s), 7.07-7.15 (5H, m), 7.2-7.5 (8H, m)

Preparation 22

(3RS)-1,1-Bis[4-(methanesulfonylamino)phenyl]-3-butylamine (77.4 mg) was obtained from (3RS)-[3,3-bis[4-(methanesulfonylamino)phenyl]-1-methylpropyl]carbamic acid benzyl ester (101 mg) by hydrogenation in the usual manner.

IR (KBr): 3425 (br s), 1635 (m), 1506 (m), 1325 (m), 1151 (s), 1103 (m), 980 (w) cm⁻¹

NMR (MeOH-d₄, δ): 1.13 (3H, d, J=6.4 Hz), 2.05 (2H, quartet, J=7.7 Hz), 2.73 (1H, quartet, J=6.5 Hz), 2.89 (6H, s), 4.06 (1H, dd, J=7.8 Hz and 14.8 Hz), 7.16 (4H, d, J=8.5 Hz), 7.26 (4H, d, J=8.5 Hz)

MS m/z: 412 (M⁺+1)

Preparation 23

To a mixture of (3RS)-3-(dibenzylamino)-1,1-bis(4-aminophenyl)-1-butanol (0.40 g), triethylamine (0.37 ml) and dichloromethane (4 ml), acetic anhydride (0.18 ml) was added at 0° C., followed by addition of additional triethylamine (0.1 ml) and acetic anhydride (0.09 ml). The reaction mixture was worked up in the usual manner to give (3RS)-3-(dibenzylamino)-1,1-bis[4-(acetylamino)phenyl]-1-butanol (0.49 g).

IR (Neat): 2300 (m), 1666 (s), 1623 (m), 1539 (s), 1514 (m), 1319 (m), 1265 (m), 1142 (w), 837 (m) cm⁻¹

NMR (CDCl₃, δ): 1.06 (3H, d, J=6.6 Hz), 2.12 (3H, s), 2.16 (3H, s), 2.0-2.2 (1H, m), 2.45-2.58 (1H, m), 3.0-3.2 (1H, m), 3.20 (2H, d, J=12.9 Hz), 3.92 (2H, d, J=13.0 Hz), 6.72 (2H, d, J=6.8 Hz), 7.09-7.36 (16H, m)

MS m/z: 536 (M⁺+1)

Preparation 24

(3RS)-3-Amino-1,1-bis[4-(acetylamino)phenyl]-1-butanol (0.23 g) was obtained from (3RS)-3-(dibenzylamino)-1,1-bis[4-(acetylamino)phenyl]-1-butanol (0.32 g) by the usual hydrogenation.

IR (KBr): 3294 (m), 1666 (s), 1623 (s), 1537 (s), 1514 (m), 1406 (m), 1321 (n), 1014 (m) cm⁻¹

NMR (MeOH-d₆, δ): 1.10-1.15 (3H, m), 2.07 (3H, s), 2.11 (3H, s), 2.5-3.1 (3H, m), 4.23 (2H, d, J=10.6 Hz), 4.55 (2H, d, J=10.6 Hz), 7.21-7.58 (8H, m)

Preparation 25

The following compound was obtained according to a similar manner to that of Example 33.

(3RS)-3-(Dibenzylamino)-1,1-bis[4-(methoxycarbonyl-amino)phenyl]-1-butanol

MS m/z: 568

Preparation 26

(3RS)-1,1-Bis[4-(methoxycarbonylamino)phenyl]-3-butylamine hydrochloride (191 mg) was obtained from (3RS)-3-(dibenzylamino)-1,1-bis(4-methoxycarbonylaminophenyl)butanol (173 mg) by hydrogenation in the usual manner.

MS m/z: 372 (M⁺+1) (free)

Preparation 27

Methanesulfonyl chloride (1.4 ml) was added dropwise to a solution of 3-amino-4-benzyloxyphenyl acetate (4.3 g) in pyridine (20 ml) under ice cooling over 10 minutes and the mixture was stirred at room temperature for a further 1 hour. To the reaction mixture was added water (100 ml) and was stirred at the same temperature for 1 hour. The precipitates were collected by filtration and dissolved into chloroform (100 ml), followed by its dryness over magnesium sulfate and its evaporation in vacuo. The residue was chromatographed (hexane-ethyl acetate) over silica gel to afford 4-benzyloxy-3-(methanesulfonylamino)phenyl acetate (1.6 g).

NMR (CDCl₃, δ): 2.27 (3H, s), 2.95 (3H, s), 5.09 (2H, s), 6.80-7.03 (3H, m), 7.25-7.45 (6H, m)

MS m/z: 336 (M⁺+1)

Preparation 28

A solution of 4-benzyloxy-3-(methanesulfonylamino)phenyl acetate (1.6 g) and potassium hydroxide (2.67 g) in methanol (10 ml) was stirred for 18 hours at room temperature. The reaction mixture was acidified with 1N hydrochloric acid to pH 5-7 and extracted with ethyl acetate. The organic layer was washed with brine, dried over magnesium sulfate, and evaporated in vacuo. The residue was chromatographed (hexane-ethyl acetate) over silica gel to afford N-(2-benzyloxy-5-hydroxyphenyl)methanesulfonamide (750 mg).

NMR (CDCl₃, δ): 2.90 (3H, s), 5.04 (2H, s), 6.58 (1H, dd, J=2.9 and 8.8 Hz), 6.80-6.90 (2H, m), 7.09 (1H, d, J=2.9 Hz), 7.30-7.50 (6H, m)

MS m/z: 294 (M⁺+1)

Preparation 29

Under nitrogen, to a solution of N-(2-benzyloxy-5-hydroxyphenyl)methanesulfonamide (740 mg) and sodium hydride (92.4 mg) in N,N-dimethylformamide (30 ml) was added (2S)-glycidyl tosylate (616 mg) at 0° C. and the mixture was stirred at the same temperature for 0.5 hour. The mixture was allowed to warm to room temperature and stirred for 2.5 hours at the same temperature. The resulting mixture was poured into 10% aqueous ammonium chloride solution, and extracted with ethyl acetate. The organic layer was washed with brine, dried over magnesium sulfate, and evaporated in vacuo. The residue was chromatographed (hexane-ethyl acetate) over silica gel to afford (2S)-3-[4-benzyloxy-3-(methanesulfonamino)phenoxy]-1,2-epoxypropane (440 mg).

NMR (CDCl₃, δ): 2.75 (1H, dd, J=2.7 and 4.9 Hz), 2.84-2.95 (4H, m), 3.30-3.37 (1H, m), 3.90 (1H, dd, J=5.8 and 11.08 Hz), 4.07-4.25 (1H, m), 5.05 (2H, s), 6.63-7.48 (9H, m)

MS m/z: 350 (M⁺+1)

Preparation 30

Under nitrogen, a solution of N-benzyl-[3,3-bis(4-methoxyphenyl)-1-methylpropyl]amine (480 mg), N-[2-benzyloxy-5-[(1R)-2-iodo-1-(triethylsilyloxy)ethyl]phenyl]-methanesulfonamide (600 mg) and N,N-diisopropylethylamine (0.74 ml) in tetrahydrofuran (6 ml) was sealed with stirring at 110° C. for 58 hours, followed by at 160° C. for 92 hours. The resulting mixture was poured into aqueous sodium hydrogen sulfite and extracted with ethyl acetate. The organic layer was washed successively with saturated aqueous sodium bicarbonate and brine, dried over anhydrous sodium sulfate, and evaporated in vacuo. The residue was purified by column chromatography on silica gel (hexane:ethyl acetate=10:1 to 5:1) to give N-[5-[(1R)-2-[N-benzyl-[(1RS)-3,3-bis(4-methoxyphenyl)-1-methylpropyl]amino]-1-(triethylsilyloxy)-ethyl]-2-(benzyloxy)phenyl]methanesulfonamide (226 mg).

NMR (CDCl₃, δ): 0.25-0.5 (6H, m), 0.7-0.95 (12H, m), 1.5-2.25 (2H, m), 2.35-2.9 (6H, m), 3.45-3.9 (8H, m), 4.25-4.4 (1H, m), 5.0-5.1 (2H, m), 6.65-7.75 (21H, m)

Preparation 31

N-Benzyl-[(1S)-3,3-bis(4-methoxyphenyl)-1-methylpropyl]-amine hydrochloride (800 mg) was dehydrochlorinated with saturated aqueous sodium bicarbonate and extracted with ethyl acetate. A mixture of the above obtained product and 10% palladium on activated carbon (50% wet, 300 mg) in methanol (10 ml) was stirred at room temperature in the presence of hydrogen at an atmospheric pressure for 5.5 hours. After filtration, the filtrate was evaporated in vacuo to give [(1S)-3,3-bis(4-methoxyphenyl)-1-methylpropyl]amine (568 mg).

NMR (CDCl₃, δ): 1.10 (3H, d, J=6.3 Hz), 1.95-2.1 (2H, m), 2.7-2.9 (1H, m), 3.76 (6H, m), 3.98 (1H, t, J=8.0 Hz), 6.75-6.9 (4H, m), 7.1-7.2 (4H, m)

Preparation 32

The following compound was obtained according to a similar manner to that of Preparation 31.

[(1R)-3,3-Bis(4-methoxyphenyl)-1-methylpropyl]amine

NMR (CDCl₃, δ): 1.19 (3H, d, J=6.3 Hz), 1.9-2.1 (2H, m), 2.7-2.85 (1H, m), 3.76 (6H, m), 3.98 (1H, t, J=7.9 Hz), 6.75-6.9 (4H, m), 7.15 (4H, d, J=8.0 Hz)

Preparation 33

A mixture of N-benzyl-[3,3-bis(4-hydroxyphenyl)-1-methylpropyl]amine (300 mg) and 10% palladium on activated carbon (50% wet, 100 mg) in methanol (5 ml) was stirred at room temperature in the presence of hydrogen at an atmospheric pressure for 4 hours. After filtration, the filtrate was evaporated in vacuo to give [3,3-bis(4-hydroxyphenyl)-1-methylpropyl]amine (230 mg).

NMR (DMSO-d₆, δ): 0.96 (3H, d, J=6.3 Hz), 1.75-1.9 (2H, m), 2.4-2.6 (1H, m), 3.87 (1H, t, J=7.9 Hz), 6.63 (4H, d, J=8.5 Hz), 7.03 (4H, d, J=8.2 Hz)

Preparation 34

The following compound was obtained according to a similar manner to that of Preparation 33.

N-Benzyl-[3,3-bis(4-methoxyphenyl)-1-methylpropyl]amine

Preparation 35

A mixture of 6-[(4-nitrophenyl)azo]pyridin-3-ol (J. Am. Chem. Soc. 1959, 81, 6049, 300 mg) and 20% palladium hydroxide on carbon (60 mg) in a mixture of acetic acid (30 ml) and methanol (30 ml) was stirred at room temperature in the presence of hydrogen at an atmospheric pressure for 70 minutes. After filtration, the filtrate was evaporated in vacuo. Under nitrogen, to a mixture of the residue in dichloromethane (10 ml) was added bis(trimethylsilyl)-acetamide (6.0 ml) at 5° C. After being stirred at room temperature for 30 minutes, to the resulting mixture was added benzyl chloroformate (0.54 ml) at 5° C., and the mixture was stirred at the same temperature for 3 hours. The resulting mixture was poured into saturated aqueous sodium bicarbonate, and extracted with ethyl acetate. The organic layer was washed with brine, dried over anhydrous magnesium sulfate, and evaporated in vacuo. To the residue was added chloroform and insoluble materials were filtered off. After the filtrate was evaporated in vacuo, the residue was purified by column chromatography on silica gel (chloroform:methanol=50:1 to 20:1), followed by crystallization from toluene-methanol to give (5-hydroxypyridin-2-yl)carbamic acid benzyl ester (159 mg).

MS m/z: 245 (M⁺+1)

Preparation 36

Under nitrogen, a suspension of sodium hydride (60% in oil, 189 mg) in N,N-dimethylformamide (20 ml) was dropwise added (5-hydroxypyridin-2-yl)carbamic acid benzyl ester (1.1 g) in N,N-dimethylformamide (12 ml) at 5° C., and the mixture was stirred at room temperature for 1 hour. To this one was added (2S)-glycidyl tosylate (1.1 g) at 5° C., and the mixture was stirred at room temperature for 7 hours. The resulting mixture was poured into saturated aqueous sodium bicarbonate, and extracted with ethyl acetate. The organic layer was washed with brine, dried over anhydrous magnesium sulfate, and evaporated in vacuo. The residue was purified by column chromatography on silica gel (chloroform:ethyl acetate=9:1) to give (2S)-3-[2-(benzyloxycarbonylamino)pyridin-5-yloxy]-1,2-epoxypropane (780 mg).

MS m/z: 301 (M⁺+1)

Preparation 37

To a suspension of but-3-enylbenzene (3 ml) and sodium bicarbonate (2.5 g) in a mixture of dichloromethane (200 ml) and water (60 ml) was added small portions of m-chloroperbenzoic acid (3.5 g) at room temperature, and the mixture was stirred at the same temperature for 4 hours. After separation, the organic layer was washed successively with saturated aqueous sodium bicarbonate and brine, dried over anhydrous magnesium sulfate and evaporated in vacuo. The residue was purified by column chromatography on silica gel (hexane:ethyl acetate=100:3) to give phenethyloxirane (970 mg).

NMR (CDCl₃, δ): 1.7-1.9 (2H, m), 2.45-2.5 (1H, m), 2.7-3.0 (4H, m), 7.1-7.4 (5H, m)

Preparation 38

Under nitrogen, to a solution of (2R)-glycidyl tosylate (3.0 g) in tetrahydrofuran (30 ml) were added N,N-diisopropylethylamine (2.5 ml) and thiophenol (1.3 ml) at 5° C., and the mixture was stirred at room temperature for 12 hours. The resulting mixture was poured into water and extracted with ethyl acetate. The organic layer was washed successively with saturated aqueous sodium bicarbonate and brine, dried over anhydrous magnesium sulfate and evaporated in vacuo. The residue was purified by column chromatography on silica gel (hexane:ethyl acetate=5:1 to 3:1) to give toluene-4-sulfonic acid (2S)-2-hydroxy-3-(phenylthio)propyl ester (3.9 g).

NMR (CDCl₃, δ): 2.44 (3H, m), 2.75-3.25 (3H, m), 3.85-4.3 (3H, m), 7.15-7.4 (7H, m), 7.7-7.8 (2H, m)

Preparation 39

Under nitrogen, to a solution of toluene-4-sulfonic acid (2S)-2-hydroxy-3-(phenylthio)propyl ester (3.9 g) in ethanol (40 ml) was added 20% sodium methoxide in ethanol (4.7 ml) at 5° C., and the mixture was stirred at the same temperature for 30 minutes. After being filtrated off to remove precipitates, the filtrate was concentrated in vacuo. The residue was dissolved into a mixture of aqueous 0.1N sodium hydroxide and diethyl ether. After separation, the organic layer was washed successively with water and brine, dried over anhydrous magnesium sulfate and evaporated in vacuo. The residue was purified by column chromatography on silica gel (hexane:ethyl acetate=20:1 to 10:1) to give (2S)-3-(phenylthio)-1,2-epoxypropane (1.5 g).

MS m/z: 167 (M++1)

### EXAMPLE 6

To a solution of 4-bromothioanisole (508 mg) in tetrahydrofuran, butyllithium (1.54M in hexane, 1.62 ml) was added at −78° C. After 30 minutes, (3RS)-3-[((2S)-2-hydroxy-3-phenoxypropyl)amino]butyric acid methyl ester (131 mg) was added and warmed to 0° C. The reaction mixture was worked up in the usual manner and purified by a silica gel column chromatography to give (2S)-1-phenoxy-3-[(3RS)-1,1-bis[4-(methylthio)phenyl]-1-hydroxy-3-butyl]amino-3-propanol (58.5 mg).

IR (Neat): 3400 (br s), 2921 (s), 1594 (s), 0.1492 (s), 1243 (s), 1093 (m), 1043 (m), 817 (s), 754 (s) cm⁻¹

NMR (CDCl₃, δ): 1.14 (3H, d, J=6.3 Hz), 2.4-2.6 (2H, m), 2.43 (3H, s), 2.46 (3H, s), 2.7-2.8 (2H, m), 2.88 (1H, dd, J=11.9 and 8.0 Hz), 3.9-4.1 (3H, m), 6.9-7.0 (3H, m), 7.1-7.4 (12H, m)

### EXAMPLE 7

The following compounds were obtained according to a similar manner to that of Example 6.

(1)-(2S)-1-Phenoxy-3-[(3RS)-1,1-bis(3-methoxyphenyl)-1-hydroxy-3-butyl]amino-2-propanol

IR (Neat): 3296 (br m), 2933 (s), 1599 (s), 1491 (s), 1464 (m), 1246 (s), 1171 (m), 1045 (s), 756 (m), 694 (m) cm⁻¹

NMR (CDCl₃+D₂O, δ): 1.09 (3H, d, J=6.3 Hz), 2.04 (1H, dd, J=11.4 and 14.2 Hz), 2.39-2.49 (2H, m), 2.6-2.8 (1H, m), 2.83 (1H, dd, J=8.3 and 11.9 Hz), 3.74 (3H, s), 3.76 (3H, s), 3.8-3.9 (2H, m), 3.9-4.1 (1H, m), 6.72-6.75 (2H, m), 6.8-7.1. (7H, m), 7.12-7.31 (4H, m)

MS m/z: 452 (M⁺+1)

(2) (2S)-1-Phenoxy-3-((3RS)-1,1-diphenyl-1-hydroxy-3-butyl)-amino-2-propanol

IR (Neat): 3292 (br m), 2925 (m), 1597 (m), 1495 (s), 1456 (m), 1244 (s), 1043 (m), 754 (s), 698 (s) cm⁻¹

MS m/z: 392 (M⁺+1)

(3) (2S)-1-Phenoxy-3-[(3RS)-1,1-bis(4-methoxyphenyl)-1-hydroxy-3-butyl]amino-2-propanol

IR (Neat): 3292 (br m), 2929 (m), 1604 (m), 1508 (s), 1460 (m), 1248 (s), 1176 (m), 1038 (m), 833 (m), 756 (m) cm⁻¹

NMR (CDCl₃, δ): 1.12 (1.5H, d, J=5.1 Hz), 1.16 (1.5H, d, J=6.2 Hz), 2.0-2.5 (2H, m), 2.6-3.2 (3H, m), 3.74 (1.5H, s), 3.75 (1.5H, s), 3.78 (3H, s), 3.8-4.2 (3H, m), 6.77-7.01 (7H, m), 7.2-7.4 (6H, m)

MS m/z: 452 (M⁺+1)

(4) (2S)-1-Phenoxy-3-[(3RS)-1,1-bis(3,4-dimethoxyphenyl)-1-hydroxy-3-butyl]amino-2-propanol

IR (Neat): 3294 (br m), 2933 (m), 1597 (w), 1510 (s), 1460 (m), 1257 (s), 1146 (m), 1028 (m), 760 (m) cm⁻¹

MS m/z: 512 (M⁺+1)

(5) (2S)-1-Phenoxy-3-[(3RS)-1,1-bis(4-methylphenyl)-1-hydroxy-3-butyl]amino-2-propanol

IR (Neat): 3400 (br s), 2923 (s), 1596 (m), 1498 (s), 1457 (s), 1243 (s), 1087 (m), 1043 (m), 817 (s), 754 (s) cm⁻¹

NMR (CDCl₃, δ): 1.13 (2H, d, J=6.2 Hz), 2.16 (3H, s), 2.31 (3H, s), 2.4-2.5 (2H, m), 2.6-2.9 (3H, m), 3.9-4.1 (3H, m), 6.9-7.4 (13H, m)

MS m/z: 420 (M⁺+1)

(6) 5-[N-Benzyl-((2S)-2-hydroxy-3-phenoxypropyl)amino]-1,1-bis(4-methoxyphenyl)-1-pentanol

IR (Neat): 3446 (br m), 2943 (m), 1604 (m), 1508 (s), 1458 (m), 1248 (s), 1176 (m), 1036 (m), 831 (m), 752 (m) cm⁻¹

NMR (CDCl₃, δ): 1.2-1.3 (2H, m), 1.4-1.7 (2H, m), 2.1-2.2 (2H, m), 2.4-2.7 (4H, m), 3.4-3.6 (1H, m), 3.78 (6H, s), 3.7-3.8 (1H, m), 3.8-3.9 (2H, m), 3.9-4.1 (1H, m), 6.77-6.97 (7H, m), 7.2-7.4 (11H, m)

MS m/z: 556 (M⁺+1)

(7) 4-[N-Benzyl-((2S)-2-hydroxy-3-phenoxypropyl)amino]-1,1-bis(4-methoxyphenyl)-1-butanol

IR (Neat): 3446 (br m), 2951 (m), 1604 (m), 1508 (s), 1458 (m), 1248 (s), 1176 (m), 1036 (s), 831 (m), 752 (m) cm⁻¹

NMR (CDCl₃, δ): 1.5-1.7 (2H, m), 2.1-2.4 (2H, m), 2.4-2.7 (4H, m), 3.4-3.5 (1H, m), 3.77 (6H, s), 3.7-3.8 (1H, m), 3.8-4.0 (2H, m), 4.1-4.3 (1H, m), 6.78-6.98 (7H, m), 7.2-7.4 (11H, m)

MS m/z: 542 (M⁺+1)

(8) (2S)-1-Phenoxy-3-[(1RS)-3,3-bis(4-methoxyphenyl)-3-hydroxy-1-(3,4-dimethoxyphenyl)propyl]amino-2-propanol

IR (Neat): 3361 (br m), 2929 (m), 1602 (m), 1512 (s), 1459 (m), 1248 (s), 1032 (s), 833 (m), 756 (m) cm⁻¹

NMR (CDCl₃, δ): 2.3-2.8 (5H, m), 3.74 (3H, s), 3.82 (3H, s), 3.86 (6H, s), 3.9-4.1 (3H, m), 6.6-7.1 (8H, m), 7.2-7.5 (8H, m)

MS m/z: 574 (M⁺+1)

(9) (2S)-1-Phenoxy-3-[(1RS)-3,3-bis(4-methoxyphenyl)-3-hydroxy-1-phenylpropyl]amino-2-propanol

IR (Neat): 3361 (br m), 2929 (m), 1603 (m), 1506 (s), 1458 (m), 1246 (s), 1176 (m), 1035 (m), 833 (m), 756 (m), 698 (m) cm⁻¹

NMR (CDCl₃, δ): 2.3-2.8 (4H, m), 3.6-3.7 (1H, m), 3.74 (3H, s), 3.82 (3H, s), 3.8-4.2 (4H, m), 6.7-7.0 (9H, m), 7.1-7.2 (2H, m), 7.2-7.4 (5H, m), 7.4-7.5 (2H, m)

MS m/z: 514 (M⁺+1)

(10) (2S)-1-Phenoxy-3-[3,3-bis(4-methoxyphenyl)-3-hydroxyphenyl]amino-2-propanol

IR (Neat): 3313 (br m), 2931 (m), 1602 (s), 1508 (s), 1462 (m), 1248 (s), 1176 (m), 1036 (m), 831 (m), 756 (m) cm⁻¹

NMR (CDCl₃+D₂O, δ): 2.3-2.5 (2H, m), 2.7-2.9 (4H, m), 3.77 (6H, s), 3.9-4.2 (4H, m), 6.80-7.01 (7H, m), 7.2-7.4 (6H, m)

MS m/z: 438 (M⁺+1)

### EXAMPLE 8

The following compounds were obtained according to a similar manner to that of Preparation 33.

(1) (2S)-1-Phenoxy-3-[5,5-bis(4-methoxyphenyl)pentyl]-amino-2-propanol hydrochloride

IR (Neat): 3322 (br m), 1602 (m), 1510 (s), 1460 (m), 1246 (s), 1178 (m), 1036 (m), 831 (m) cm⁻¹

NMR (MeOH-d₄, δ): 1.2-1.4 (2H, m), 1.6-1.8 (2H, m), 2.04 (2H, quartet, J=7.5 Hz), 2.99 (2H, t, J=8.0 Hz), 3.09-3.22 (2H, m), 3.73 (6H, s), 3.6-3.85 (1H, m), 3.9-4.0 (2H, m), 4.1-4.3 (1H, m), 6.84 (4H, d, J=8.7 Hz), 6.9-7.0 (3H, m), 7.14 (4H, d, J=8.6 Hz), 7.28 (2H, t, J=7.9 Hz)

MS m/z: 450 (M⁺+1) (free)

(2) (2S)-1-Phenoxy-3-[4,4-bis(4-methoxyphenyl)butyl]amino-2-propanol hydrochloride

IR (Neat): 3355 (br m), 1602 (m), 1510 (s), 1460 (m), 1246 (s) 1178 (m), 1036 (m), 831 (m) cm⁻¹

NMR (MeOH-d₄, 5): 1.6-1.8 (2H, m), 2.08 (1H, quartet, J=7.9 Hz), 2.55 (1H, quartet, J=7.6 Hz), 3.0-3.2 (4H, m), 3.73 (6H, s), 3.7-4.3 (4H, m), 6.8-7.0 (7H, m), 7.1-7.4 (6H, m)

MS m/z: 436 (M⁺+1) (free)

### EXAMPLE 9

A mixture of (2S)-1-phenoxy-3-[(3RS)-1,1-bis[4-(methylthio)phenyl]-1-hydroxy-3-butyl]amino-2-propanol (47 mg), water (2 ml), methanol (3 ml) and OXONE® (potassium peroxymonosulfate) (180 mg) was stirred at room temperature overnight and worked up in the usual manner to give (2S)-1-phenoxy-3-[(3RS)-1,1-bis(4-methanesulfonylphenyl)-1-hydroxy-3-butyl]amino-2-propanol (52 mg).

IR (Neat): 3521 (br m), 2927 (m), 1595 (m), 1494 (s), 1309 (s), 1244 (m), 1149 (s), 1091 (m), 958 (m), 771 (s), 694 (m) cm⁻¹

NMR (CDCl₃, δ): 1.19 (3H, d, J=6.3 Hz), 2.3-2.8 (4H, m), 2.9 (1H, m), 3.00 (3H, s), 3.05 (3H, s), 3.9-4.1 (3H, m), 6.9-7.1 (3H, m), 7.2-7.4 (1H, m), 7.5-7.74 (5H, m), 7.8-7.9 (4H, m)

MS m/z: 548 (M⁺+1)

### EXAMPLE 10

A mixture of (2S)-3-phenoxy-1,2-epoxypropane (40 mg), 3-amino-3-(3,4-dimethoxyphenyl)propionic acid methyl ester acetate (80 mg), triethylamine (0.5 ml) and methanol (3 ml) was heated under reflux, evaporated and purified by silica gel column chromatography (hexane:ethyl acetate:methanol=1:1:0.07) to give (3RS)-3-((2S)-2-hydroxy-3-phenoxypropyl)-amino-3-(3,4-dimethoxyphenyl)propionic acid methyl ester (92 mg).

IR (Neat): 2925 (m), 1738 (s), 1597 (m), 1514 (s), 1460 (m), 1263 (m), 1138 (m), 1027 (s), 758 (m) cm⁻¹

NMR (CDCl₃, δ): 2.6-2.8 (4H, m), 3.67 (3H, s), 3.87 (6H, s), 3.9-4.0 (2H, m), 4.0-4.1 (2H, m), 6.8-7.0 (7H, m), 7.26 (1H, t, J=8.9 Hz)

MS m/z: 390 (M⁺+1)

### EXAMPLE 11

The following compound was obtained according to a similar manner to that of Example 10.

(2S)-1-Phenoxy-3-[1,1-bis(4-methoxyphenyl)-3-methyl-3-butyl]amino-2-propanol

IR (Neat): 3350 (br m), 2962 (m), 1606 (m), 1508 (s), 1460 (m), 1248 (s), 1178 (m), 1036 (m), 829 (m), 756 (m) cm⁻¹

NMR (CDCl₃, δ):1.03 (3H, s), 1.05 (3H, s), 2.22 (2H, d, J=6.8 Hz), 2.55 (1H, dd, J=7.0 and 11.7 Hz), 2.68 (1H, dd, J=3.6 and 11.7 Hz), 3.73 (6H, s), 3.8-3.9 (3H, m), 4.04 (1H, t, J=6.7 Hz), 6.77-6.99 (7H, m), 7.1-7.4 (6H, m)

MS m/z: 450 (M⁺+1)

### EXAMPLE 12

A mixture of (2S)-3-phenoxy-1,2-epoxypropane (0.12 g), 1-[2,2-bis(4-methoxyphenyl)ethyl]cyclopentylamine (0.24 g) and methanol (5 ml) was heated under reflux, evaporated and purified by silica gel column chromatography (hexane:ethyl acetate:methanol=1:1:0.07) to give N-((2S)-2-hydroxy-3-phenoxypropyl)-[1-[2,2-bis(4-methoxyphenyl)ethyl]-cyclopentyl]amine (40.9 mg).

IR (Neat): 2954 (m), 1606 (w), 1510 (s), 1460 (m), 1246 (m), 1176 (m), 1038 (s), 825 (m), 754 (m) cm⁻¹

NMR (CDCl₃, δ): 1.2-1.8 (8H, m), 2.25 (2H, t, J=6.8 Hz), 2.46 (1H, dd, J=6.9 and 12.0 Hz), 2.61 (1H, dd, J=4.4 and 12.0 Hz), 3.73 (6H, s), 3.8-3.9 (2H, m), 4.0-4.1 (2H, m), 6.78 (4H, d, J=8.2 Hz), 6.9-7.0 (3H, m), 7.19 (4H, d, J=8.7 Hz), 7.24-7.33 (2H, m)

MS m/z: 476 (M⁺+1)

### EXAMPLE 13

The following compounds were obtained according to a similar manner to that of Example 12.

(1) (1R)-1-(3-Pyridyl)-2-[[(3RS)-1,1-bis(4-methoxyphenyl)-3-butyl]amino]ethanol

MS m/z: 407 (M⁺+1)

(2) (2S)-1-(3-Pyridyloxy)-3-[(3RS)-1,1-bis(4-methoxyphenyl)-3-butyl]amino-2-propanol dihydrochloride

MS m/z: 437 (M⁺+1) (free)

(3) (2S)-1-(1H-Indol-4-yloxy)-3-[3,3-bis(4-methoxyphenyl)-propyl]amino-2-propanol

MS m/z: 461 (M⁺+1)

(4) (2RS)-1-(2-Oxo-2,3-dihydro-1H-benzimidazol-4-yloxy)-3-[(3RS)-1,1-bis(4-methoxyphenyl)-3-butyl]amino-2-propanol

MS m/z: 492 (M⁺+1)

(5) (2R)-3-[4-Benzyloxy-3-(methanesulfonylamino)phenyl]-1-[(3RS)-1,1-bis(4-methoxyphenyl)-3-butyl]amino-2-propanol

(6) (2S)-1-Phenoxy-3-[(3RS)-1,1-bis[4-(methanesulfonyl-amino)phenyl]-3-butyl]amino-2-propanol

IR (KBr): 3440 (br s), 1603 (m), 1508 (m), 1325 (m), 1242 (m), 1151 (s), 1103 (m), 974 (m), 758 (m) cm⁻¹

NMR (CDCl₃, δ): 1.11 (3H, d, J=6.2 Hz), 2.0-2.2 (2H, m), 2.2-2.9 (3H, m), 2.97 (6H, s), 3.9-4.0 (3H, br s), 4.1-4.2 (1H, m), 6.88-7.00 (4H, m), 7.10-7.33 (9H, m)

MS m/z: 562 (M⁺+1)

(7) (2S)-1-Phenoxy-3-[(3RS)-1,1-bis[4-(acetylamino)phenyl]-1-hydroxy-3-butyl]amino-2-propanol

MS m/z: 506 (M⁺+1)

(8) (2S)-1-Phenoxy-3-[(3RS)-1,1-bis[4-(acetylamino)phenyl]-3-butyl]amino-2-propanol

MS m/z: 490 (M⁺+1)

### EXAMPLE 14

A mixture of (1S)-1-phenoxy-3-[3,3-bis(4-methoxyphenyl)-3-hydroxypropyl]amino-2-propanol (93 mg), p-toluenesulfonic acid hydrate (53 mg) and toluene was heated under reflux for 1.5 hours, evaporated and purified (preparative TLC, silica gel, 10% methanol-dichloromethane) to give (1S)-1-phenoxy-3-[3,3-bis(4-methoxyphenyl)-2-propenyl]amino-2-propanol (80.5 mg).

IR (Neat): 3359 (br m), 1604 (s), 1510 (s), 1460 (m), 1248 (s), 1176 (m), 1034 (m), 835 (m), 756 (m), 686 (m) cm⁻¹

NMR (CDCl₃+D₂O, δ): 2.9-3.1 (2H, m), 3.58 (2H, d, J=7.0 Hz), 3.77 (3H, s), 3.82 (3H, s), 3.9-4.0 (2H, m), 4.2-4.3 (1H, m), 6.07 (1H, t, J=7.1 Hz), 6.73-7.29 (13H, m)

### EXAMPLE 15

To a mixture of (2S)-1-phenoxy-3-[3,3-bis(4-methoxyphenyl)-3-hydroxypropyl]amino-2-propanol (34 mg), triethylsilane (0.5 ml) and dichloromethane (1 ml), trifluoroacetic acid (0.1 ml) was added dropwise at room temperature. The reaction mixture was worked up immediately in the usual manner and purified by silica gel preparative TLC (eluent; 10% methanol/dichloromethane) to give (2S)-1-phenoxy-3-[3,3-bis(4-methoxyphenyl)propyl]amino-2-propanol trifluoroacetate (21 mg).

IR (Neat): 3400 (br m), 2933 (m), 1680 (s), 1604 (m), 1508 (s), 1248 (s), 1203 (m), 1180 (s), 1134 (m), 1036 (m), 829 (m), 756 (m) cm⁻¹

NMR (CDCl₃, δ): 2.3-2.5 (2H, m), 2.7-2.9 (2H, m), 2.9-3.1 (2H, m), 3.74 (6H, s), 3.8-4.0 (3H, m), 4.1-4.3 (1H, m), 6.7-6.9 (6H, m), 6.95 (2H, t, J=7.4 Hz), 7.10 (4H, d, J=8.5 Hz), 7.26 (1H, t, J=7.9 Hz)

MS m/z: 422 (M⁺+1)

### EXAMPLE 16

A mixture of (2S)-3-phenoxy-1,2-epoxypropane (0.11 g), D-alanine bis(4-methoxyphenyl)methylamide (0.25 g) and methanol (4 ml) was heated under reflux overnight, evaporated and purified by silica gel column chromatography to give N-((2S)-2-hydroxy-3-phenoxypropyl)-D-alanine [bis(4-methoxyphenyl)methyl]amide (217 mg).

IR (KBr): 3290 (s), 1643 (s), 1606 (m), 1512 (s), 1642 (m), 1250 (s), 1176 (m), 1034 (s), 831 (w), 812 (m), 752 (m) cm⁻¹

NMR (CDCl₃, δ): 1.27 (3H, d, J=7.2 Hz), 2.66 (1H, dd, J=3.8 and 12.0 Hz), 2.80 (1H, dd, J=7.6 and 12.0 Hz), 3.24 (1H, quartet, J=6.9 Hz), 3.74 (3H, s), 3.78 (3H, s), 3.8-4.0 (3H, m), 6.14 (1H, d, J=8.6 Hz), 6.8-6.9 (6H, m), 6.98 (1H, t, J=7.3 Hz), 7.13 (4H, dd, J=2.0 and 8.6 Hz), 7.29 (2H, t, J=7.4 Hz), 7.71 (1H, d, J=8.6 Hz)

MS m/z: 465 (M⁺+1)

### EXAMPLE 17

To a suspension of lithium aluminum hydride (10 mg) in tetrahydrofuran (0.5 ml), a solution of N-((2S)-2-hydroxy-3-phenoxypropyl)-D-alanine [bis(4-methoxyphenyl)methyl]amide (52.4 mg) in tetrahydrofuran was added dropwise at 0° C. under a flow of nitrogen. The reaction mixture was heated under reflux. After 2 hours, additional lithium aluminum hydride (50 mg) was added to the reaction mixture under a flow of nitrogen at 0° C. The reaction mixture was heated under reflux for 2.5 hours, worked up in the usual manner and purified (preparative TLC, 10% methanol-ethyl acetate) to give (2S)-1-phenoxy-3-[(2R)-1-[[bis(4-methoxyphenyl)methyl]-amino]-2-propyl]amino-2-propanol (31.4 mg).

IR (Neat): 3316 (br s), 2931 (m), 1606 (m), 1508 (s), 1458 (m), 1292 (s), 1174 (m), 1036 (s), 820 (m), 756 (m) cm⁻¹

NMR (CDCl₃, δ): 1.06 (3H, d, J=6.3 Hz), 2.46 (1H, dd, J=8.7 and 12.0 Hz), 2.62 (1H, dd, J=4.2 and 12.0 Hz), 2.80 (2H, d, J=4.7 Hz), 3.76 (6H, s), 3.9-4.1 (3H, m), 4.71 (1H, s), 6.82 (4H, dd, J=2.0 and 6.7 Hz), 6.9-7.0 (3H, m), 7.2-7.3 (6H, m)

MS m/z: 451 (M⁺+1)

### EXAMPLE 18

A mixture of (2R)-2-[4-benzyloxy-3-(methanesulfonylamino)phenyl]-2-(triethylsilyloxy)-1-iodoethane (156 mg), [3,3-bis(4-methoxyphenyl)propyl]amine (75 mg), N,N-diisopropylethylamine (0.19 ml) and dimethylacetamide (0.75 ml) was heated at 110° C. overnight and worked up in the usual manner. The crude product was treated with 4N hydrogen chloride in ethyl acetate (2 ml), worked up in the usual manner and purified by preparative TLC (10% methanol-dichloromethane) to give (1R)-1-[4-benzyloxy-3-(methanesulfonylamino)phenyl]-2-[3,3-bis(4-methoxyphenyl)-propyl]aminoethanol (47 mg).

IR (Neat): 3310 (br m), 1608 (w), 1510 (s), 1460 (m), 1329 (m), 1248 (s), 1157 (s), 1120 (s), 1034 (m), 818 (m), 739 (m) cm⁻¹

NMR (CDCl₃, δ): 2.16 (2H, quartet, J=7.1 Hz), 2.5-2.7 (3H, m), 2.81 (1H, dd, J=3.6 and 12.2 Hz), 2.90 (3H, s), 3.79 (6H, s), 3.95 (1H, t, J=7.9 Hz), 4.55 (1H, dd, J=3.5 and 8.9 Hz), 5.09 (2H, s), 6.81 (4H, d, J=8.6 Hz), 6.95 (1H, d, J=8.5 Hz), 7.13 (5H, d, J=8.6 Hz), 7.35 (5H, s), 7.47 (1H, d, J=2.0 Hz)

MS m/z: 591 (M⁺+1)

### EXAMPLE 19

The following compound was obtained according to a similar manner to that of Example 18.

(1R)-1-[4-Benzyloxy-3-(methanesulfonylamino)phenyl]-2-[[(3RS)-1,1-bis[4-(methoxycarbonylamino)phenyl]-3-butyl]-amino]ethanol

### EXAMPLE 20

(1R)-1-[4-Benzyloxy-3-(methanesulfonylamino)phenyl]-2-[[3,3-bis(4-methoxyphenyl)propyl]amino]ethanol (35 mg) was hydrogenated by the usual manner to give (1R)-1-[4-hydroxy-3-(methanesulfonylamino)phenyl]-2-[[3,3-bis(4-methoxyphenyl)-propyl]amino]ethanol (19.3 mg).

IR (KBr): 3430 (br m), 1608 (w), 1510 (s), 1319 (m), 1304 (m), 1248 (s), 1153 (m), 1034 (m), 825 (m) cm⁻¹

NMR (CDCl₃, δ): 2.1-2.3 (2H, m), 2.5-2.7 (2H, m), 2.7-2.9 (2H, m), 2.90 (3H, s), 3.74 (6H, s), 3.83 (1H, t, J=7.8 Hz), 4.5-4.7 (1H, m), 6.79 (5H, d, J=8.3 Hz), 7.01 (1H, d, J=8.1 Hz), 7.13 (4H, d, J=8.4 Hz), 7.13 (1H, br s)

MS m/z: 501 (M⁺+1)

### EXAMPLE 21

A mixture of (2S)-3-(4-benzyloxy-3-nitrophenoxy)-1,2-epoxypropane (197 mg), N-benzyl-[3,3-bis(4-methoxyphenyl)-propyl]amine (236 mg) and ethanol (3 ml) was heated under reflux for 12 hours. Iron powder, ammonium chloride and water were added to the reaction mixture and heating was continued for 1 hour. The reaction mixture was filtrated and worked up in the usual manner to give (2S)-1-(3-amino-4-benzyloxyphenoxy)-3-[N-benzyl-[3,3-bis(4-methoxyphenyl)-propyl]amino]-2-propanol (412.7 mg).

NMR (CDCl₃, δ): 2.1-2.3 (2H, m), 2.4-2.7 (4H, m), 3.50 (1H, d, J=14 Hz), 3.75 (6H, s), 3.7-4.0 (5H, m), 5.01 (2H, s), 6.15-6.4 (2H, m), 6.71-6.80 (5H, m), 7.03-7.08 (4H, m), 7.2-7.4 (1øH, m)

MS m/z: 633 (M⁺+1)

### EXAMPLE 22

The following compounds were obtained according to a similar manner to that of Example 21.

(1) (2S)-1-(3-Amino-4-benzyloxyphenoxy)-3-[N-benzyl-[4,4-bis(4-methoxyphenyl)butyl]amino]-2-propanol

NMR (CDCl₃, δ): 1.45 (2H, quintet, J=7.5 Hz), 1.93 (2H, quintet), 2.3-2.6 (4H, m), 3.44 (1H, d, J=13.5 Hz), 3.69-4.1 (4H, m), 3.76 (6H, s), 5.00 (2H, s), 6.17 (1H, dd, J=2.9 and 8.8 Hz), 6.31 (1H, d, J=2.8 Hz), 6.73 (1H, d, J=8.8 Hz), 6.79 (4H, d, J=8.7 Hz), 7.07 (4H, d, J=7.7 Hz), 7.2-7.4 (10H, m)

MS m/z: 647 (M⁺+1)

(2) (1RS)-1-(3-Amino-4-benzyloxyphenyl)-2-[N-benzyl-[4,4-bis(4-methoxyphenyl)butyl]amino]ethanol

NMR (CDCl₃, δ): 1.4-1.6 (2H, m), 1.8-2.1 (2H, m), 2.4-2.7 (4H, m), 3.41 (1H, d, J=13.5 Hz), 3.76 (6H, s), 3.7-3.9 (2H, m), 4.51 (1H, t), 5.05 (2H, s), 6.50-6.65 (2H, m), 6.75-6.85 (5H, m), 7.05-7.15 (4H, m), 7.2-7.5 (10H, m)

MS m/z: 617 (M⁺+1)

(3) (2S)-1-Phenoxy-3-[[3,3-bis(4-ethoxyphenyl)propyl]-amino]-2-propanol

IR (Neat): 3305 (br m), 1604 (m), 1510 (s), 1392 (w), 1300 (w), 1246 (s), 1176 (m), 1047 (s), 822 (m), 756 (m) cm⁻¹

NMR (MeOH-d₆, δ): 1.34 (6H, t, J=7.0 Hz), 2.2-2.4 (2H, m), 2.6-2.9 (4H, m), 3.97 (4H, quartet, J=7.0 Hz), 3.9-4.1 (4H, m), 6.80 (4H, d, J=8.6 Hz), 6.89-7.0 (3H, m), 7.14 (4H, d, J=8.6 Hz), 7.26 (2H, t, J=7.9 Hz)

MS m/z: 450 (M⁺+1)

### EXAMPLE 23

To a mixture of (2S)-1-(3-amino-4-benzyloxyphenoxy)-3-[N-benzyl-[3,3-bis(4-methoxyphenyl)propyl]amino]-2-propanol (59 mg), pyridine (0.1 ml) and dichloromethane (1 ml), methanesulfonyl chloride (27 μl) were added at 0° C. and stirred for 30 minutes. The reaction mixture was worked up in the usual manner. The crude product was hydrogenated in the usual manner to give (2S)-1-[4-hydroxy-3-(methanesulfonylamino)phenoxy]-3-[[3,3-bis(4-methoxyphenyl)-propyl]amino]-2-propanol (17.2 mg).

IR (KBr): 3440 (br s), 1610 (w), 1510 (s), 1460 (m), 1325 (m), 1248 (s), 1176 (m), 1151 (m), 1115 (w), 1034 (m), 816 (m) cm⁻¹

NMR (MeOH-d₄, δ): 2.1-2.3 (2H, m), 2.5-2.8 (4H, m), 2.91 (3H, s), 3.74 (6H, s), 3.8-4.1 (4H, m), 6.6-6.7 (1H, m), 6.7-6.9 (5H, m), 6.97 (1H, d, J=2.7 Hz), 7.1-7.2 (4H, m)

MS m/z: 531 (M⁺+1)

### EXAMPLE 24

The following compounds were obtained according to a similar manner to that of Example 23.

(1) (1R)-1-[4-Hydroxy-3-(methanesulfonylamino)phenyl]-2-[[3,3-bis[4-[(methoxycarbonyl)amino]phenyl]propyl]-amino]ethanol

MS m/z: 587 (M⁺+1)

(2) (2S)-1-[(4-Hydroxy-3-(methanesulfonylamino)phenoxy]-3-[[3,3-bis[4-[(methoxycarbonyl)amino]phenyl]propyl]-amino]-2-propanol

MS m/z: 617 (M⁺+1)

(3) (2S)-1-[4-Hydroxy-3-(methanesulfonylamino)phenoxy]-3-[4,4-bis(4-methoxyphenyl)butyl]amino-2-propanol

IR (KBr): 3480 (br m), 1612 (m), 1512 (s), 1460 (m), 1321 (w), 1248 (s), 1178 (m), 1113 (m), 1034 (m), 826 (m) cm⁻¹

NMR (MeOH-d₄, δ): 1.4-1.6 (2H, m), 1.9-2.1 (2H, m), 2.6-2.8 (4H, m), 2.89 (3H, s), 3.73 (6H, s), 3.8-4.1 (4H, m), 6.61 (1H, dd, J=2.9 and 8.8 Hz), 6.76 (1H, d, J=8.5 Hz), 6.80 (4H, d, J=8.6 Hz), 6.96 (1H, d, J=2.9 Hz), 7.13 (4H, d, J=8.6 Hz)

MS m/z: 545 (M⁺+1)

(4) (1RS)-1-[4-Hydroxy-3-(methanesulfonylamino)phenyl]-2-[[4,4-bis(4-methoxyphenyl)butyl]amino]ethanol

IR (KBr): 3420 (br m), 1560 (m), 1512 (s), 1321 (m), 1248 (s), 1153 (m), 1113 (w), 1034 (m), 817 (m) cm⁻¹

NMR (MeOH-d₄, δ): 1.4-1.7 (2H, m), 1.9-2.1 (2H, m), 2.7-2.9 (4H, m), 2.90 (3H, s), 3.73 (6H, s), 3.7-3.9 (1H, m), 4.6-4.8 (1H, m), 6.81 (4H, d, J=8.7 Hz), 6.85 (1H, m), 7.05 (1H, m), 7.14 (4H, d, J=8.6 Hz), 7.34 (1H, s)

MS m/z: 515 (M⁺+1)

### EXAMPLE 25

To a solution of (2S)-1-phenoxy-3-[N-benzyl-[3,3-bis(4-methoxyphenyl)propyl]amino]-2-propanol (47 mg) and dichloromethane (1 ml), 1M boron tribromide-dichloromethane solution (0.28 ml) was added at −78° C. The reaction mixture was stirred at 0° C. for 50 minutes and worked up in the usual manner to give (2S)-1-phenoxy-3-[N-benzyl-[3,3-bis(4-hydroxyphenyl)propyl]amino]-2-propanol (44 mg).

MS m/z: 484 (M⁺+1)

### EXAMPLE 26

To a mixture of (2S)-1-phenoxy-3-[N-benzyl-[3,3-bis(4-hydroxyphenyl)propyl]amino]-2-propanol (40 mg), N,N-diisopropylethylamine (43 μl) and dichloromethane (1 ml), trifluoromethanesulfonic anhydride (31 μl) was added at −78° C. The reaction mixture was worked up in the usual manner. A mixture of the obtained crude product, palladium acetate (5.6 mg), 1,3-bis(diphenylphosphino)propane (10.2 mg), triethylamine (46 μl), N,N-dimethylformamide (1 ml) and methanol (0.5 ml) was stirred at 100° C. under carbon monoxide atmosphere (1 atm) for 3.5 hours, worked up in the usual manner and purified by preparative TLC (hexane:ethyl acetate=3:1) to give (2S)-1-phenoxy-3-[N-benzyl-[3,3-bis[4-(methoxycarbonyl)phenyl]propyl]amino]-2-propanol (21 mg).

MS m/z: 568 (M++1)

### EXAMPLE 27

N-[5-[(2S)-3-[N-Benzyl-[(1RS)-3,3-bis(4-hydroxyphenyl)-1-methylpropyl]amino]-2-hydroxypropoxy]-2-benzyloxyphenyl]-methanesulfonamide (120 mg) and 10% palladium on activated carbon (50% wet, 30 mg) in methanol (10 ml) was stirred at room temperature in the presence of hydrogen at an atmospheric pressure for 3 hours, and filtered. The filtrate was evaporated in vacuo and treated with 4N hydrogen chloride in ethyl acetate to afford N-[5-[(2S)-3-[[(1RS)-3,3-bis(4-hydroxyphenyl)-1-methylpropyl]amino]-2-hydroxypropoxy]-2-hydroxyphenyl]methanesulfonamide hydrochloride (50 mg).

MS m/z: 516 (M⁺+1) (free)

### EXAMPLE 28

The following compound was obtained according to a similar manner to that of Example 27.

N-[5-[(2S)-3-[[(1RS)-3,3-Bis(4-methoxyphenyl)-1-methylpropyl]amino]-2-hydroxypropoxy]-2-hydroxyphenyl]-methanesulfonamide hydrochloride

MS m/z: 544 (M⁺+1) (free)

### EXAMPLE 29

(2S)-1-Phenoxy-3-[N-benzyl-[3,3-bis[4-(methoxycarbonyl)-phenyl]propyl]amino]-2-propanol (14 mg) was treated with sodium hydroxide in the usual manner and hydrogenated in the usual manner to give (2S)-1-phenoxy-3-[3,3-bis(4-carboxyphenyl)propyl]amino-2-propanol (12 mg).

MS m/z: 450 (M⁺+1)

### EXAMPLE 30

A mixture of (2S)-3-phenoxy-1,2-epoxypropane (0.36 g), N-benzyl-[3,3-bis[4-[(methoxycarbonyl)amino]phenyl]propyl]-amine (0.97 g) and ethanol (10 ml) was heated under reflux for 12 hours and cooled to room temperature. To the reaction mixture, 10% palladium on charcoal (50% wet, 0.4 g), 4N hydrogen chloride in 1,4-dioxane (1.1 ml) and methanol (5 ml) was added. The mixture was stirred under hydrogen (1 atm) for 3.5 hours, filtrated, diluted with ethyl acetate, neutralized by washing with aqueous sodium bicarbonate solution and the organic layer was evaporated. The crude product was purified by silica gel column chromatography(dichloromethane:methanol:concentrated ammonia water=20:1:0.05) to give (2S)-1-phenoxy-3-[[3,3-bis[4-[(methoxycarbonyl)amino]phenyl]propyl]amino]-2-propanol, which was converted to the corresponding hydrochloride salt (0.71 g) in a usual manner.

IR (KBr): 3400 (br m), 1711 (s), 1599 (m), 1537 (s), 1317 (m), 1238 (s), 1072 (m), 758 (m) cm⁻¹

NMR (MeOH-d₄, δ): 2.3-2.5 (2H, m), 2.9-3.3 (4H, m), 3.65 (3H, s), 3.71 (3H, s), 3.9-4.00 (3H, m), 4.1-4.3 (1H, m), 6.91-6.98 (3H, m), 7.18-7.39 (10H, m)

MS m/z: 508 (M⁺+1) (free)

### EXAMPLE 31

The following compound was obtained according to a similar manner to that of Example 30.

(2S)-1-(4-Hydroxyphenoxy)-3-[(3RS)-1,1-bis(4-methoxyphenyl)-3-butyl]amino-2-propanol

MS m/z: 452 (M⁺+1)

### EXAMPLE 32

The following compound was obtained according to a similar manner to that of Preparation 16.

(2S)-1-Phenoxy-3-[N-benzyl-[(3RS)-1,1-bis(4-aminophenyl)-1-hydroxy-3-butyl]amino]-2-propanol

MS m/z: 512 (M⁺+1)

### EXAMPLE 33

To a mixture of (2S)-1-phenoxy-3-[N-benzyl-[(3RS)-1,1-bis(4-aminophenyl)-1-hydroxy-3-butyl]amino]-2-propanol (100 mg), pyridine (48 μl) and dichloromethane (2 ml), methyl chlorocarbonate (33 μl) was added at 0° C. The reaction mixture was worked up in the usual manner to give (2S)-1-phenoxy-3-[N-benzyl-[(3RS)-1,1-bis[4-[(methoxycarbonyl)-amino]phenyl]-1-hydroxy-3-butyl]amino]-2-propanol (125 mg).

MS m/z: 628 (M⁺+1)

### EXAMPLE 34

The following compound was obtained according to a similar manner to that of Example 33.

(2S)-1-Phenoxy-3-[N-benzyl-[(3RS)-1,1-bis[4-[N-methyl-(methoxycarbonyl)amino]phenyl]-1-hydroxy-3-butyl]amino]-2-propanol

MS m/z: 656 (M⁺+1)

### EXAMPLE 35

(2S)-1-Phenoxy-3-[N-(benzyl-[(3RS)-1,1-bis[4-[(methoxycarbonyl)amino]phenyl]-1-hydroxy-3-butyl]amino]-2-propanol (99 mg) was hydrogenated in the usual manner to give (2S)-1-phenoxy-3-[(3RS)-1,1-bis[4-[(methoxycarbonyl)amino]-phenyl]-3-butyl]amino-2-propanol (58 mg).

MS m/z: 522 (M⁺+1)

### EXAMPLE 36

The following compounds were obtained according to a similar manner to that of Preparation 18.

(1) (2S)-1-Phenoxy-3-[(3RS)-1,1-bis[4-[(ethoxycarbonyl)-amino]phenyl]-3-butyl]amino-2-propanol hydrochloride

MS m/z: 550 (M⁺+1) (free)

(2) (2S)-1-Phenoxy-3-[(3RS)-1,1-bis[4-[(trifluoroacetyl)-amino]phenyl]-1-hydroxy-3-butyl]amino-2-propanol hydrochloride

MS m/z: 614 (M⁺+1) (free)

(3) (2S)-1-Phenoxy-3-[(3RS)-1,1-bis[4-(propionylamino)-phenyl]-3-butyl]amino-2-propanol

MS m/z: 518 (M++1)

### EXAMPLE 37

To a mixture of (2S)-1-phenoxy-3-[N-benzyl-[(3RS)-1,1-bis(4-aminophenyl)-1-hydroxy-3-butyl]amino]-2-propanol (120 mg), acetic acid (3 ml) and water (0.6 ml), potassium cyanate (77 mg) was added. The reaction mixture was worked up in the usual manner to give (2S)-1-phenoxy-3-[N-benzyl-[(3RS)-1,1-bis(4-ureidophenyl)-1-hydroxy-3-butyl]amino]-2-propanol (65 mg).

MS m/z: 598 (M⁺+1)

### EXAMPLE 38

Formic acid (650 μl) and acetic anhydride (540 μl) were mixed and started at room temperature for 30 minutes. The mixture was added to a solution of (2S)-1-phenoxy-3-[N-benzyl-[(3RS)-1,1-bis(4-aminophenyl)-1-hydroxy-3-butyl]-amino]-2-propanol (325 mg) in dichloromethane (2 ml) at 0° C., warmed to room temperature and worked up in the usual manner. The crude product was stirred with potassium carbonate (0.62 g) in methanol (4 ml) at room temperature for 4 hours and worked up in the usual manner to give (2S)-1-phenoxy-3-[N-benzyl-[(3RS)-1,1-bis[4-(formylamino)phenyl]-1-hydroxy-3-butyl]amino]-2-propanol (342.4 mg).

MS m/z: 568 (M⁺+1)

### EXAMPLE 39

To a mixture of lithium aluminum hydride (0.1 g) and tetrahydrofuran (1 ml), a solution of (2S)-1-phenoxy-3-[N-benzyl-[(3RS)-1,1-bis[4-(formylamino)phenyl]-1-hydroxy-3-butyl]amino]-2-propanol (280 mg) in tetrahydrofuran (2 ml) was added dropwise at 0° C. The reaction mixture was stirred for 2.5 hours and worked up in the usual manner to give (2S)-1-phenoxy-3-[N-benzyl-[(3RS)-1,1-bis[4-(methylamino)phenyl]-1-hydroxy-3-butyl]amino]-2-propanol (273 mg).

MS m/z: 540 (M⁺+1)

### EXAMPLE 40

(2S)-1-Phenoxy-3-[(3RS)-1,1-bis[4-[N-methyl-(methoxycarbonyl)amino]phenyl]-1-hydroxy-3-butyl]amino-2-propanol (30 mg) was obtained from (2S)-1-phenoxy-3-[N-benzyl-[(3RS)-1,1-bis[4-[N-methyl-(methoxycarbonyl)amino]-phenyl]-1-hydroxy-3-butyl]amino]-2-propanol (60 mg) by the usual hydrogenation.

MS m/z: 566 (M⁺+1)

### EXAMPLE 41

(1R)-1-[4-Hydroxy-3-(methanesulfonylamino)phenyl]-2-[[(3RS)-1,1-bis[4-[(methoxycarbonyl)amino]phenyl]-3-butyl]-amino]ethanol (14.3 mg) was obtained from (1R)-1-[4-bezyloxy-3-(methanesulfonylamino)phenyl]-2-[[(3RS)-1 μl-bis[4-[(methoxycarbonyl)amino]phenyl]-3-butyl]amino]ethanol (46.1 mg) by hydrogenation in the usual manner.

MS m/z: 601 (M⁺+1)

### EXAMPLE 42

The following compound was obtained according to a similar manner to that of Example 41.

(2R)-3-[4-Hydroxy-3-(methanesulfonylamino)phenyl]-1-[(3RS)-1,1-bis(4-methoxyphenyl)-3-butyl]amino-2-propanol (5.0 mg)

MS m/z: 529 (M⁺+1)

### EXAMPLE 43

A mixture of (R)-(4-benzyloxy-3-nitrophenyl)oxirane (34.4 mg), N-benzyl-[3,3-bis[4-(methoxycarbonylamino)phenyl]-propyl]amine (56.7 mg) and ethanol (2 ml) was heated under reflux for 12 hours. Iron powder, ammonium chloride and water was added to the reaction mixture and heating was continued for 1 hour. The reaction mixture was filtrated and worked up in the usual manner to give crude (1R)-1-(3-amino-4-benzyloxyphenyl)-2-[N-benzyl-[3,3-bis[4-[(methoxycarbonyl)amino]phenyl]propyl]amino]ethanol (111.7 mg).

MS m/z: 689 (M⁺+1)

### EXAMPLE 44

The following compound was obtained according to a similar manner to that of Example 43.

(2S)-1-(3-Amino-4-benzyloxyphenoxy)-3-[N-benzyl-[3,3-bis[4-[(methoxycarbonyl)amino]phenyl]propyl]amino]-2-propanol

MS m/z: 719

### EXAMPLE 45

Under nitrogen, to a solution of 4,4-bis(4-methoxyphenyl)-2-butanone (187 mg) and (1RS)-2-amino-1-(2-methylpyridin-6-yl)ethanol (100 mg) prepared from 6-methylpyridin-2-carboxaldehyde and trimethylsilylcyanide catalized with zinc iodide followed by reduction with lithium aluminum hydride, in 1,2-dichloroethane (10 ml) was added sodium triacetoxyborohydride (257 mg) at room temperatue, and the mixture was stirred at the same temperature for 24 hours. The resulting mixture was poured into saturated aqueous sodium bicarbonate solution, and extracted with ethyl acetate. The organic layer was washed with brine, dried over magnesium sulfate, and evaporated in vacuo. The residue was treated with 4N hydrogen chloride in 1,4-dioxane to afford (1RS)-2-[[(1RS)-3,3-bis(4-methoxyphenyl)-1-methylpropyl]-amino]-1-(6-methylpyridin-2-yl)ethanol (140 mg) dihydrochloride.

MS m/z: 421 (M⁺+1) (free)

### EXAMPLE 46

Under nitrogen, to a solution of (2S)-3-[4-benzyloxy-3-(methanesulfonylamino)phenoxy]-1,2-epoxypropane (198 mg) and N-benzyl-[(1RS)-3,3-bis(4-hydroxyphenyl)-1-methylpropyl]amine (20.0 mg) in methanol (20 ml) was refluxed for 18 hours. The resulting mixture was poured into saturated aqueous sodium bicarbonate solution, and extracted with ethyl acetate. The organic layer was washed with brine, dried over magnesium sulfate, and evaporated in vacuo. The residue was chromatographed (hexane-ethyl acetate) over silica gel to afford N-[5-[(2S)-3-[N-benzyl-[(1RS)-3,3-bis(4-hydroxyphenyl)-1-methylpropyl]amino]-2-hydroxypropoxy]-2-(benzyloxy)phenyl]methanesulfonamide (120 mg).

MS m/z: 696 (M⁺+1)

### EXAMPLE 47

Under nitrogen, to a solution of (2S)-3-[4-benzyloxy-3-(methanesulfonylamino)phenoxy]-1,2-epoxypropane (200 mg) and N-benzyl-[(1RS)-3,3-bis(4-methoxyphenyl)-1-methylpropyl]amine (163 mg) in dichloromethane (10 ml) was added ytterbium(III) trifluoromethanesulfonate (1.0 g) at room temperature, and the mixture was stirred at the same temperature for 3 hours. The resulting mixture was poured into saturated aqueous sodium bicarbonate solution, and extracted with ethyl acetate. The organic layer was washed with brine, dried over magnesium sulfate, and evaporated in vacuo. The residue was chromatographed (hexane-ethyl acetate) over silica gel to afford N-[5-[(2S)-3-[N-benzyl-[(1RS)-3,3-bis(4-methoxyphenyl)-1-methylpropyl]amino]-2-hydroxypropoxy]-2-(benzyloxy)phenyl]methanesulfonamide (50 mg).

MS m/z: 724 (M⁺+1)

### EXAMPLE 48

Under nitrogen, to a solution of N-benzyl-[3,3-bis(4-hydroxyphenyl)-1-methylpropyl]amine (300 mg) and phenethyloxirane (130 mg) in a mixture of ethyl acetate (5 ml) and tetrahydrofuran (5 ml) was added ytterbium(III) trifluoromethanesulfonate (110 mg) at room temperature, and the mixture was stirred at the same temperature for 96 hours. The resulting mixture was poured into saturated aqueous sodium bicarbonate and extracted with ethyl acetate. The organic layer was washed with brine, dried over anhydrous magnesium sulfate, and evaporated in vacuo. The residue was purified by column chromatography on silica gel (chloroform:methanol=20:1) to give 1-[N-benzyl-[3,3-bis(4-hydroxyphenyl)-1-methylpropyl]amino]-4-phenyl-2-butanol (240 mg).

NMR (CDCl₃, δ): 0.95-1.10 (3H, m), 1.45-2.9 (9H, m), 3.2-3.75 (3H, m), 3.75-3.9 (1H, m), 6.55-6.8 (4H, m), 6.85-7.3 (14H, m)

### EXAMPLE 49

The following compound was obtained according to a similar manner to that of Example 48.

(2S)-1-[N-Benzyl-[(1RS)-3,3-bis(4-hydroxyphenyl)-1-methylpropyl]amino]-3-(phenylthio)-2-propanol

NMR (CDCl₃, δ): 0.85-1.1 (3H, m), 1.7-3.1 (7H, m), 3.3-3.75 (3H, m), 3.75-3.9 (1H, m), 6.55-6.75 (4H, m), 6.8-7.25 (14H, m)

### EXAMPLE 50

Under nitrogen, to a solution of N-[5-[(1R)-2-[N-benzyl-[(1RS)-3,3-bis(4-methoxyphenyl)-1-methylpropyl]amino]-1-(triethylsilyloxy)ethyl]-2-(benzyloxy)phenyl]methanesulfonamide (221 mg) in tetrahydrofuran (3 ml) were added acetic acid (63 μl) and tetrabutylammonium fluoride (1M solution in tetrahydrofuran, 0.68 ml) at room temperature, and the mixture was stirred at the same temperature for 4.5 hours. The resulting mixture was poured into saturated aqueous sodium bicarbonate and extracted with ethyl acetate. The organic layer was washed with brine, dried over anhydrous magnesium sulfate, and evaporated in vacuo. The residue was purified by column chromatography on silica gel (hexane:ethyl acetate=2:1) to give N-[5-[(1R)-2-[N-benzyl-[(1RS)-3,3-bis(4-methoxyphenyl)-1-methylpropyl]amino]-1-hydroxyethyl]-2-(benzyloxy)phenyl]methanesulfonamide (164 mg).

NMR (CDCl₃, δ): 0.95-1.1 (3H, m), 1.7-2.85 (5H, m), 2.88 (3H, m), 3.35-4.05 (8H, m), 4.25-4.5 (1H, m), 5.08 (2H, m), 6.7-7.5 (21H, m)

### EXAMPLE 51

A mixture of N-[5-[(1R)-2-[N-benzyl-[(1RS)-3,3-bis(4-methoxyphenyl)-1-methylpropyl]amino]-1-hydroxyethyl]-2-(benzyloxy)phenyl]methanesulfonamide (149 mg) and 10% palladium on activated carbon (50% wet, 50 mg) in methanol (5 ml) was stirred at room temperature in the presence of hydrogen at an atmospheric pressure for 6 hours. After filtration, the filtrate was evaporated in vacuo, followed by treatment with 4N hydrogen chloride in ethyl acetate to give N-[5-[(1R)-2-[(1RS)-3,3-bis(4-methoxyphenyl)-1-methylpropyl]-amino-1-hydroxyethyl]-2-hydroxyphenyl]methanesulfonamide hydrochloride (90 mg).

NMR (DMSO-d₆, δ): 1.1-1.35 (3H, m), 1.9-2.2 (1H, m), 2.55-3.1 (7H, m), 3.70 (6H, m), 3.95-4.1 (1H, m), 4.7-4.9 (1H, m), 6.8-7.4 (11H, m)

### EXAMPLE 52

The following compounds were obtained according to a similar manner to that of Example 51.

(1) 1-[3,3-Bis(4-hydroxyphenyl)-1-methylpropyl]amino-4-phenyl-2-butanol hydrochloride

NMR (CD₃OD, δ): 1.1-1.5 (3H, m), 1.7-1.9 (2H, m), 1.95-2.2 (1H, m), 2.45-3.2 (6H, m), 3.6-4.0 (1H, m), 6.5-6.8 (4H, m), 7.0-7.35 (9H, m)

(2) (2S)-1-Benzenesulfonyl-3-[(1RS)-3,3-bis(4-hydroxyphenyl)-1-methylpropyl]amino-2-propanol hydrochloride

NMR (CD₃OD, δ): 1.25-1.4 (3H, m), 1.95-2.2 (1H, m), 2.45-2.7 (1H, m), 2.9-3.55 (5H, m), 3.85-4.0 (1H, m), 4.25-4.4 (1H, m), 6.65-6.85 (4H, m), 7.05-7.2 (4H, m), 7.6-7.8 (3H, m), 7.95-8.05 (2H, m)

(3) (2S)-1-Phenoxy-3-[(3RS)-1 μl-bis(4-ureidophenyl)-3-butyl]amino-2-propanol hydrochloride

MS m/z: 492 (M⁺+1) (free)

### EXAMPLE 53

Under nitrogen, a solution of [(1S)-3,3-bis(4-methoxyphenyl)-1-methylpropyl]amine (0.55 g), N-[2-benzyloxy-5-[(1R)-2-iodo-1-(triethylsilyloxy)ethyl]phenyl]-methanesulfonamide (1.1 g) and N,N-diisopropylethylamine (1.4 ml) in N,N-dimethylacetamide (5 ml) was stirred at 110° C. for 24 hours. The resulting mixture was poured into saturated aqueous sodium bicarbonate and extracted with ethyl acetate. The organic layer was washed successively with water and brine, dried over anhydrous sodium sulfate and evaporated in vacuo. Under nitrogen, to the residue in ethyl acetate (10 ml) was added 4N hydrogen chloride in ethyl acetate (2 ml) at 5° C., and the mixture was stirred at room temperature for 45 minutes. The resulting mixture was poured into saturated aqueous sodium bicarbonate and extracted with ethyl acetate. The organic layer was washed with brine, dried over anhydrous magnesium sulfate and evaporated in vacuo. The residue was purified by column chromatography on silica gel (chloroform:methanol=50:1 to 20:1) to give N-[2-benzyloxy-5-[(1R)-2-[(1S)-3,3-bis(4-methoxyphenyl)-1-methylpropyl]-amino-1-hydroxyethyl]phenyl]methanesulfonamide (0.65 g).

NMR (CDCl₃, δ): 1.09 (3H, d, J=6.3 Hz), 1.85-2.3 (2H, m), 2.35-2.6 (2H, m), 2.9-3.2 (4H, m), 3.76 (6H, s), 4.0-4.1 (1H, m), 4.45-4.6 (1H, m), 5.10 (2H, m), 6.82 (4H, d, J=8.1 Hz), 6.96 (1H, d, J=8.5 Hz), 7.1-7.2 (5H, m), 7.35-7.5 (6H, m)

### EXAMPLE 54

The following compounds were obtained according to a similar manner to that of Example 53.

(1) N-[2-Benzyloxy-5-[(1R)-2-[(1R)-3,3-bis(4-methoxyphenyl)-1-methylpropyl]amino-1-hydroxyethyl]phenyl]-methanesulfonamide

NMR (CDCl₃, δ): 1.08 (3H, d, J=6.2 Hz), 1.9-2.2 (2H, m), 2.5-2.85 (3H, m), 2.90 (3H, s), 3.76 (6H, s), 4.03 (1H, t, J=8.2 Hz), 4.47 (1H, dd, J=3.6 and 8.5 Hz), 5.10 (2H, s), 6.8-6.9 (4H, m), 6.96 (1H, d, J=8.5 Hz), 7.1-7.2 (5H, m), 7.35-7.5 (6H, m)

(2) N-[2-Benzyloxy-5-[(1R)-2-[(1RS)-3,3-bis(4-hydroxyphenyl)-1-methylpropyl]amino-1-hydroxyethyl]phenyl]-methanesulfonamide

NMR (DMSO-d₆, δ): 1.0-1.1 (3H, m), 1.7-1.95 (1H, m), 2.1-2.85 (4H, m), 2.90 (3H, s), 3.8-3.95 (1H, m), 4.5-4.6 (1H, m), 5.17 (2H, s), 6.6-6.75 (4H, m), 6.95-7.2 (6H, m), 7.25-7.6 (6H, m)

### EXAMPLE 55

A mixture of N-[2-(benzyloxy)-5-[(1R)-2-[(1S)-3,3-bis(4-methoxyphenyl)-1-methylpropyl]amino-1-hydroxyethyl]phenyl]-methanesulfonamide (620 mg) and 10% palladium on activated carbon (50% wet, 300 mg) in methanol (10 ml) was stirred at room temperature in the presence of hydrogen at an atmospheric pressure for 7.5 hours. After filtration, the filtrate was evaporated in vacuo. The residue was purified by column chromatography on silica gel (chloroform:methanol=20:1 to 10:1), followed by treatment with 4N hydrogen chloride in ethyl acetate to give N-[5-[(1R)-2-[(1S)-3,3-bis(4-methoxyphenyl)-1-methylpropyl]amino-1-hydroxyethyl]-2-hydroxyphenyl]methanesulfonamide hydrochloride (290 mg)

NMR (DMSO-d₆, δ): 1.15-1.4 (3H, m), 1.85-2.2 (1H, m), 2.4-3.2 (7H, m), 3.70 (6H, s), 3.95-4.1 (1H, m), 4.7-4.9 (1H, m), 6.7-7.4 (11H, m)

### EXAMPLE 56

The following compounds were obtained according to a similar manner to that of Example 54.

(1) N-[5-[(1R)-2-[(1R)-3,3-Bis(4-methoxyphenyl)-1-methylpropyl]amino-1-hydroxyethyl]-2-hydroxyphenyl]methanesulfonamide hydrochloride

NMR (DMSO-d₆, δ): 1.15-1.4 (3H, m), 1.9-2.15 (1H, m), 2.4-3.15 (7H, m), 3.70 (6H, m), 3.95-4.1 (1H, m), 4.75-4.9 (1H, m), 6.8-7.4 (11H, m)

(2) N-[5-[(1R)-2-[[3,3-Bis(4-hydroxyphenyl)-1-methylpropyl]-amino]-1-hydroxyethyl]-2-hydroxyphenyl]-methanesulfonamide hydrochloride

NMR (DMSO-d₆, δ): 1.15-1.3 (3H, m), 1.85-2.1 (1H, m), 2.55-3.2 (7H, m), 3.8-4.0 (1H, m), 4.7-4.9 (1H, m), 6.6-6.75 (4H, m), 6.9-7.3 (7H, m)

(3) (2S)-1-(6-Aminopyridin-3-yloxy)-3-[[(1RS)-3,3-bis(4-methoxyphenyl)-1-methylpropyl]amino]-2-propanol trihydrochloride, starting from the objective compound of Example 57.

NMR (DMSO-d₆, δ): 1.05-1.4 (3H, m), 1.9-2.2 (1H, m), 2.5-3.2 (4H, m), 3.55-3.85 (7H, m), 3.85-4.3 (3H, m), 6.9-7.4 (9H, m), 7.5-7.9 (2H, m)

### EXAMPLE 57

A mixture of [3,3-bis(4-methoxyphenyl)-1-methylpropyl]-amine and (2S)-3-[2-(benzyloxycarbonylamino)pyridin-5-yloxy]-1,2-epoxypropane (98 mg) in methanol (5 ml) was refluxed for 19 hours. After removal of the solvent in vacuo, the residue was purified by column chromatography on silica gel (chloroform:methanol=30:1 to 20:1) to give [5-[(2S)-3-[(1RS)-3,3-bis(4-methoxyphenyl)-1-methylpropyl]amino-2-hydroxypropoxy]pyridin-2-yl]carbamic acid benzyl ester (110 mg).

NMR (CDCl₃, δ): 1.1-1.2 (3H, m), 1.7-2.3 (2H, m), 2.45-2.6 (2H, m), 2.7-2.75 (1H, m), 3.76 (6H, s), 3.85-3.95 (3H, m), 4.0-4.1 (1H, m), 5.22 (2H, s), 6.8 (4H, d, J=8.6 Hz), 7.1-7.45 (10H, m), 7.9-7.95 (2H, m)

### EXAMPLE 58

To a solution of (2S)-1-[N-benzyl-[(1RS)-3,3-bis(4-hydroxyphenyl)-1-methylpropyl]amino]-3-phenylthio-2-propanol (300 mg) in methanol (10 ml) was added OXONE® (potassium peroxymonosulfate) (710 mg) in water (2 ml) at room temperature, and the mixture was stirred at the same temperature for 4 hours. The resulting mixture was poured into a mixture of ethyl acetate and water, and was made basic with saturated aqueous sodium bicarbonate. After separation, the organic layer was washed with brine, dried over anhydrous magnesium sulfate and evaporated in vacuo. The residue was purified by column chromatography on silica gel (chloroform:methanol=20:1) to give (2S)-1-benzenesulfonyl-3-[N-benzyl-[(1RS)-3,3-bis(4-hydroxyphenyl)-1-methylpropyl]amino]-2-propanol (220 mg).

NMR (CDCl₃, δ): 0.9-1.1 (3H, m), 1.75-2.3 (2H, m), 2.35-2.7 (3H, m), 2.9-3.25 (2H, m), 3.3-4.0 (4H, m), 6.65-6.8 (4H, m), 6.9-7.35 (9H, m), 7.5-7.7 (3H, m), 7.75-7.9 (2H, m)

### EXAMPLE 59

A mixture of (2S)-1-phenoxy-3-[N-benzyl-[3,3-bis[4-(methoxycarbonyl)phenyl]propyl]amino]-2-propanol (103 mg), methanol (2 ml), 1,4-dioxane (2 ml) and 1N aqueous sodium hydroxide solution (1 ml) was stirred at 50° C. for 2 hours. The reaction mixture was acidified with 3N hydrochloric acid (1 ml) and worked up in a usual manner to give (2S)-1-phenoxy-3-[N-benzyl-[3,3-bis(4-carboxyphenyl)propyl]amino]-2-propanol (75.1 mg).

### EXAMPLE 60

A mixture of (2S)-1-phenoxy-3-[N-benzyl-[3,3-bis(4-carboxyphenyl)propyl]amino]-2-propanol (75 mg), diphenylphosphoryl azide (96 mg), triethylamine (58 μl), toluene (1 ml) and 1,4-dioxane (1 ml) was stirred at 50° C. for 0.5 hour, then at 100° C. for 45 minutes. Methanol (1 ml) was added to the reaction mixture, and the heating was continued for 15 hours. The reaction mixture was worked up in a usual manner followed by purification by preparative thin-layer chromatography to afford (2S)-1-phenoxy-3-[N-benzyl-[3,3-bis[4-[(methoxycarbonyl)amino]phenyl]propyl]amino]-2-propanol (21.5 mg).

MS m/z: 598 (M⁺+1)

### EXAMPLE 61

(2S)-1-Phenoxy-3-[N-benzyl-[3,3-bis[4-[(methoxy-carbonyl)amino]phenyl]propyl]amino]-2-propanol (18.8 mg) was hydrogenated in a usual manner to give (2S)-1-phenoxy-3-[[3,3-bis[4-[(methoxycarbonyl)amino]phenyl]propyl]amino]-2-propanol (8.1 mg).

IR (KBr): 1710 (s), 1601 (m), 1537 (s), 1315 (w), 1238 (s), 1070 (m) cm⁻¹

NMR (MeOH-d₄, δ): 2.2-2.3 (2H, m), 2.6-2.9 (4H, m), 3.72 (6H, s), 3.9-4.1 (4H, m), 6.9-7.0 (3H, m), 7.2-7.4 (10H, m)

MS m/z: 508 (M⁺+1)

1. A compound of the general formula [I] 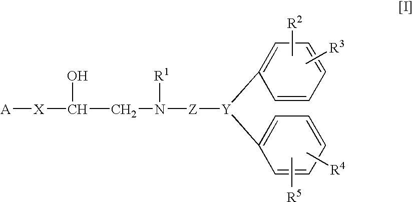
wherein
A is a heterocyclic group or aryl, each of which may have 1 to 3 same or different substituent(s) selected from a group consisting of halogen, hydroxy, amino, lower alkyl, lower alkylsulfonylamino, phenyl(lower)alkoxy and phenyl(lower)alkoxycarbonylamino,
—X— is bond, —CH₂—, —CH₂—CH₂—, —NH—CH₂—, —O—CH₂—, —S—CH₂—, —SO—CH₂— or —SO₂—CH₂—, 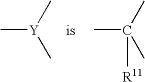
(in which R¹ is hydrogen, hydroxy, lower alkoxy or acyloxy) and 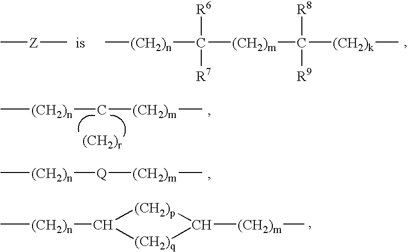
—(CH₂)ₙ CH═CH(CH₂)ₘ— or —(CH₂)ₙ—C═C—(CH₂)ₘ— (in which
-Q- is —O—, —S—, —SO—, —SO₂—, 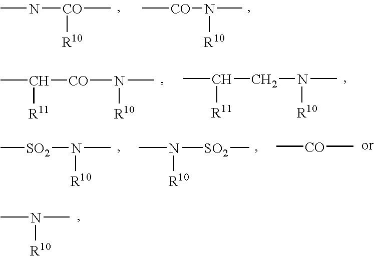
wherein R¹⁰ is hydrogen or lower alkyl, and R¹¹ is lower alkyl,
R⁶, R⁷, R⁸ and R⁹ are each independently hydrogen, hydroxy, lower alkyl, lower alkenyl, lower alkoxy, lower alkoxy(lower)alkyl or aryl which may have 1 to 3 lower alkoxy,
n, m and k is each independently 0 to 6,
p is 0 to 4,
q is 1 to 4, and
r is 2 to 7) and 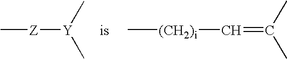
(in which i is 0 to 6),
R¹ is hydrogen or an amino protective group, and
R², R³, R⁴ and R⁵ are each independently hydrogen;
lower alkyl; lower alkylthio; lower alkylsulfonyl;
hydroxy; lower alkoxy;
amino; lower alkylamino; acylamino; N-(lower alkyl)acylamino; carboxy; lower alkoxycarbonyl;
carbamoyl optionally substituted with one or two lower alkyl; hydroxy(lower)alkyl; lower alkoxy(lower)alkyl;
N-acylamino(lower)alkyl; N-(lower alkyl)-N-acylamino(lower)alkyl; carboxy(lower)alkyl;
lower alkoxycarbonyl(lower)alkyl; carbamoyl(lower)alkyl optionally substituted with one or two lower alkyl; or 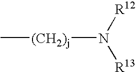
(in which R and R³ are each independently hydrogen or lower alkyl, or R¹² and R¹³ may be bonded to form a lower alkylene chain, and j is 0 to 6),
and a salt thereof.

2. A compound of claim 1, wherein
A is pyridyl, indolyl, 2-oxo-2,3-dihydro-1H-benzimidazolyl or phenyl, each of which may have 1 to 3 same or different substituent(s) selected from a group of hydroxy, amino, lower alkyl, lower alkylsulfonylamino, phenyl(lower)alkoxy and phenyl(lower)alkoxycarbonylamino,
—X— is bond, —CH₂—, —CH₂—CH₂—, —O—CH₂— or —SO₂—CH₂—, 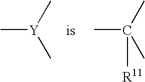
(in which R¹¹ is hydrogen or hydroxy) and
-Z- is 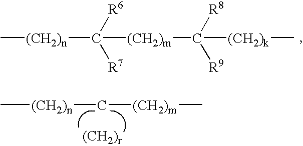
or
(CH₂)ₙ-Q-(CH₂)ₘ— (in which
-Q- is 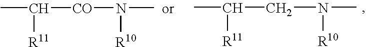
wherein
R¹⁰ is hydrogen or lower alkyl and
R¹¹ is lower alkyl,
R⁶, R⁷, R⁸ and R⁹ are each independently hydrogen, lower alkyl or aryl which may have 1 to 3 lower alkoxy,
n, m and k is each independently 0 to 6, and
r is 2 to 7) and 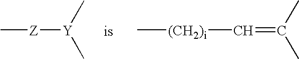
(in which i is 0 to 6),
R¹ is hydrogen or ar(lower)alkyl, and
R², R³, R⁴ and R⁵ are each independently hydrogen;
lower alkyl; lower alkylthio; lower alkylsulfonyl;
hydroxy; lower alkoxy; amino; lower alkylamino;
acylamino; N-(lower alkyl)acylamino; carboxy; or lower alkoxycarbonyl.

3. A compound of claim 2, wherein
A is pyridyl, indolyl or phenyl, each of which may have 1 to 3 same or different substituent(s) selected from a group of hydroxy, amino, lower alkyl, lower alkylsulfonylamino, phenyl(lower)alkoxy and phenyl(lower)alkoxycarbonylamino,
—X— is bond, —CH₂—, —CH₂—CH₂—, —O—CH₂— or —SO₂—CH₂—, 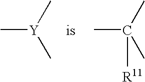
(in which R¹¹ is hydrogen or hydroxy) and
-Z- is 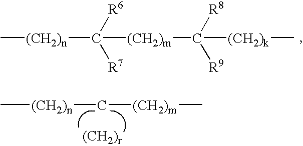
or
(CH₂)ₙ-Q-(CH₂)ₘ— (in which
-Q- is 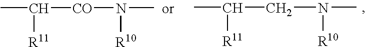
wherein
R¹⁰ is hydrogen or lower alkyl and
R¹¹ is lower alkyl,
R⁶, R⁷, R⁸ and R⁹ are each independently hydrogen, lower alkyl or phenyl which may have 1 to 3 lower alkoxy,
n, m and k is each independently 0 or 1, and
r is 2 to 7) and 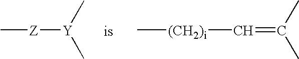
(in which i is 0 or 1),
R¹ is hydrogen or phenyl(lower)alkyl, and
R², R³, R and R⁵ are each independently hydrogen;
lower alkyl; lower alkylthio; lower alkylsulfonyl;
hydroxy; lower alkoxy; amino; lower alkylamino;
lower alkoxycarbonylamino; lower alkylsulfonylamino; lower alkanoylamino; ureido;
trifluoroacetylamino; N-(lower alkyl)-[(lower)alkoxycarbonyl]amino; carboxy; or lower alkoxycarbonyl.

4. A compound of claim 3, whrein
A is phenyl which may have 1 to 3 same or different substituent(s) selected from a group of hydroxy, amino, lower alkylsulfonylamino and phenyl(lower)alkoxy,
—X— is bond, —CH₂—, —CH₂—CH₂—, —O—CH₂— or —SO₂—CH₂—, 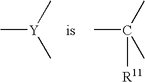
(in which R¹¹ is hydrogen or hydroxy),
-Z- is 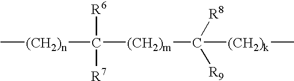
(in which
R⁶, R R⁸ and R⁹ are each independently hydrogen, lower alkyl or phenyl which may have 1 to 3 lower alkoxy,
n, m and k is each independently 0 or 1),
R¹ is hydrogen or phenyl(lower)alkyl, and
R², R³, R⁴ and R⁵ are each independently hydrogen;
lower alkyl; lower alkylthio; lower alkylsulfonyl;
hydroxy; lower alkoxy; amino; lower alkylamino;
lower alkoxycarbonylamino; lower alkylsulfonylamino; lower alkanoylamino; ureido;
trifluoroacetylamino; N-(lower alkyl)-[(lower)alkoxycarbonyl]amino; carboxy; or lower alkoxycarbonyl.

5. A compound of claim 4, wherein
A is phenyl which may have 1 or 2 same or different substituent(s) selected from a group consisting of hydroxy, amino and lower alkylsulfonylamino,
—X— is bond or —O—CH₂—, 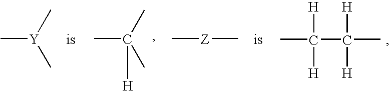
R¹ is hydrogen, and
R², R R⁴ and R⁵ are each independently hydrogen, lower alkoxy or lower alkoxycarbonylamino.

6. A compound of claim 5, which is
(2S)-1-[4-hydroxy-3-(methanesulfonylamino)phenoxy]-3-[[3,3-bis(4-methoxyphenyl)propyl]amino]-2-propanol;
(1R)-1-[4-hydroxy-3-(methanesulfonylamino)phenyl]-2-[[3,3-bis(4-methoxyphenyl)propyl]amino]ethanol;
(2S)-1-phenoxy-3-[[3,3-bis[4-[(methoxycarbonyl)amino]-phenyl]propyl]amino]-2-propanol;
or a salt thereof.

7. A process for preparing a compound of claim 1, or a salt thereof,
which comprises,
(i) reacting a compound [II] of the formula 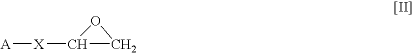
wherein A and X are each as defined in claim 1, with a compound [III] of the formula 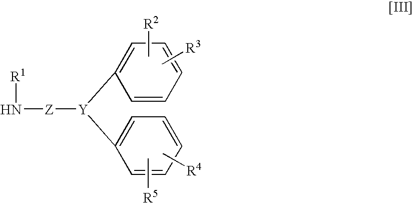
wherein Y, Z, R¹, R², R³, R⁴ and R⁵ are each as defined in claim 1, or a salt thereof, to give a compound [I] of the formula 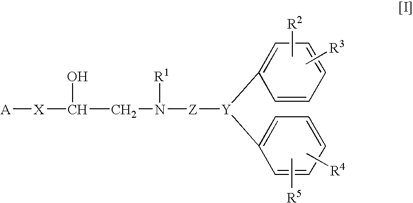
wherein A, X, Y, Z, R¹, R², R³, R⁴ and R⁵ are each as defined in claim 1,
or a salt thereof, or
(ii) subjecting a compound [Ia] of the formula 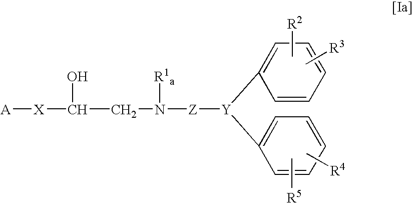
wherein A, X, Y, Z, R², R³, R⁴ and R⁵ are each as defined in claim 1, and
Rₐ¹ is an amino protective group,
or a salt thereof, to elimination reaction of the amino protective group, to give a compound [Ib] of the formula 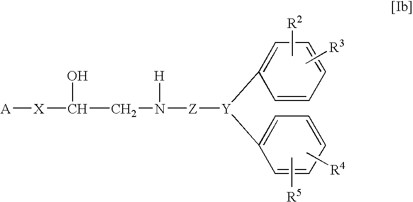
wherein A, X, Y, Z, R², R³, R⁴ and R⁵ are each as defined in claim 1,
or a salt thereof.

8. A pharmaceutical composition which comprises, as an active ingredient, a compound of claim 1 or a pharmaceutically acceptable salt thereof in admixture with pharmaceutically acceptable carriers or excipients.

9. Use of a compound of claim 1 or a pharmaceutically acceptable salt thereof for the manufacture of a medicament.

10. A compound of claim 1 or a pharmaceutically acceptable salt thereof for use as a medicament.

11. A method for the prophylactic and/or the therapeutic treatment of pollakiuria or urinary incontinence which comprises administering a compound of claim 1 or a pharmaceutically acceptable salt thereof to a human being or an animal.
